(19) United States
(12) Patent Application Publication
(10) Pub. No.: US 2025/0009680 A1
(43) Pub. Date: Jan. 9, 2025

(54) REGENERATIVE GROWTH FACTORS FOR NERVE REPAIR, PREPARATION PROCESSES OF THE SAME, AND TREATMENT METHODS USING THE SAME

(71) Applicant: The Johns Hopkins University, Baltimore, MD (US)

(72) Inventors: Hai-Quan MAO, Baltimore, MD (US); Sami TUFFAHA, Baltimore, MD (US); Chenhu QIU, Baltimore, MD (US); Sashank REDDY, Baltimore, MD (US); Yicheng ZHANG, Baltimore, MD (US); Thomas HARRIS, Baltimore, MD (US); Philip HANWRIGHT, Baltimore, MD (US); Karim A. SARHANE, Baltimore, MD (US); Nicholas VON GUIONNEAU, Baltimore, MD (US); Erica LEE, Baltimore, MD (US)

(21) Appl. No.: 18/705,652

(22) PCT Filed: Nov. 1, 2022
(86) PCT No.: PCT/US2022/079040

§ 371 Date: Apr. 29, 2024

Related U.S. Application Data
(60) Provisional application No. 63/274,364, filed on Nov. 1, 2021.

(51) Int. Cl. A61K 9/51; A61K 9/50; A61K 38/30; A61P 25/00

(52) U.S. Cl. CPC A61K 9/5192; A61K 9/5031; A61K 9/5161; A61K 38/30; A61P 25/00

(57) ABSTRACT

Methods for preparing one or more nanoparticles comprising an amphiphilic block copolymer having a polyelectrolyte complex comprising one or more therapeutic small proteins and a counter ion polymer encapsulated therein and their use for treating peripheral nerve injuries are disclosed.

### TECHNICAL FIELD

The presently disclosed subject matter relates generally to nanoparticles comprising an amphiphilic block copolymer having a polyelectrolyte complex comprising one or more therapeutic small proteins and a counter ion polymer encapsulated therein, methods of making same, and methods of their use for treating peripheral nerve injuries.

### BACKGROUND

Peripheral nerve injuries (PNIs) are common with an estimated incidence of 68,000 major PNIs occurring each year in the United States alone. Taylor et al., 2008. Debilitating motor and sensory deficits persist in a majority of cases despite optimal surgical management. Bruyns et al., 2003. Poor outcomes primarily result from the prolonged period of latency prior to reinnervation of distal targets. Carlson et al., 1996. Following surgical repair, axons must regenerate across long distances at a relatively slow rate of approximately 1 mm per day to reinnervate distal targets. Throughout this lengthy process, denervated skeletal muscle undergoes progressive, irreversible atrophy that limits the potential for meaningful functional recovery when reinnervation occurs. Grisnell and Keating, 2014. Moreover, in the absence of direct axonal contact, Schwann cells (SCs) within the distal nerve track senesce and lose the capacity to provide structural and neurotrophic support to regenerating axons, further delaying and limiting end-organ reinnervation. Sulaiman and Gordon, 2000; Gordon et al., 2011. There are currently no therapeutics available, however, to improve functional recovery in patients with PNI.

### SUMMARY

In some aspects, the presently disclosed subject matter provides a method for preparing one or more nanoparticles comprising an amphiphilic block copolymer having a polyelectrolyte complex comprising one or more therapeutic small proteins and a counter ion polymer encapsulated therein, the method comprising:
(a) assembling the polyelectrolyte complex comprising a therapeutic small protein and a counterion polymer, wherein the counter ion polymer has a charge enabling it to bind electrostatically to the one or more therapeutic small proteins; and
(b) encapsulating the polyelectrolyte complex with an amphiphilic block copolymer through a flash nanoprecipitation (FNP) process.

In certain aspects, the therapeutic small protein comprises a growth factor or a proteoglycan. In particular aspects, the therapeutic small protein comprises insulin-like growth factor 1 (IGF-1) or agrin.

In certain aspects, the counter ion polymer is selected from dextran sulfate (DS), heparin (heparin sulfate), hyaluronic acid, and combinations thereof. In particular aspects, the counter ion comprises dextran sulfate.

In certain aspects, the amphiphilic block copolymer is selected from poly(ethylene glycol)-block-poly(D,L-lactic acid) (PEG-b-PDLLA), poly(ethylene glycol)-block-poly(ε-caprolactone) (PEG-b-PCL), poly(ethylene glycol)-block-poly(lactic-co-glycolic acid) (PEG-b-PLGA) and combinations thereof. In particular aspects, the amphiphilic block copolymer comprises poly(ethylene glycol)-block-poly(ε-caprolactone) (PEG-b-PCL). In more particular aspects, the amphiphilic block copolymer comprises PEG10k-b-PCL40k.

In particular aspects, the therapeutic small protein comprises IGF-1, the counter ion polymer comprises dextran sulfate, and the amphiphilic block polymer comprises poly(ethylene glycol)-block-poly(ε-caprolactone) (PEG-b-PCL).

In certain aspects, the flash nanoprecipitation (FNP) method comprises a multi-inlet vortex mixer. In particular aspects, the multi-inlet vortex mixer comprises four inlets. In more particular aspects, the one inlet of the multi-inlet vortex mixer is in fluid communication with a source of the polyelectrolyte complex, one inlet of the multi-inlet vortex mixer is in fluid communication with a source of the amphiphilic block polymer, and two inlets of the multi-inlet vortex mixer are in fluid communication with a source of water.

In certain aspects, the assembling of the polyelectrolyte complex is conducted at a pH that is below the isoelectric point of the protein, under which condition the therapeutic protein carries net positive charges.

In certain aspects, the assembling of the polyelectrolyte complex is conducted at a pH from about 1 to about 5. In particular aspects, the pH is about 4.

In certain aspects, the one or more nanoparticles have a hydrodynamic size that decreases with flow rate. In certain aspects, the one or more nanoparticles have a poly dispersity index that decreases with flow rate. In certain aspects, each jet has a flow rate ranging from about 1 mL/min to about 60 mL/min. In particular aspects, the flow rate of each jet is about 5 mL/min.

In certain aspects, an encapsulation efficiency increases with increasing amphiphilic block polymer:therapeutic small protein mass ratio. In particular aspects, the polyelectrolyte complex has a mass ratio of counter ion polymer to therapeutic small protein of between about 0.1 to about 10. In yet more particular aspects, the mass ratio is about 0.2.

In certain aspects, the amphiphilic block copolymer is in a water-miscible solvent. In particular aspects, the water-miscible solvent is selected from acetonitrile (ACN), dimethylformamide (DMF), dimethyl sulfoxide (DMSO), and tetrahydrofuran (THF). In more particular aspects, the water-miscible solvent comprises acetonitrile or dimethylformamide. In yet more particular aspects, the water-miscible solvent comprises acetonitrile. In certain aspects, the polyelectrolyte complex is suspended in dimethyl sulfoxide (DMSO).

In certain aspects, the one or more nanoparticles have a ratio of the amphiphilic block copolymer to the small therapeutic protein of about 0.1 to 10. In certain aspects, the one or more nanoparticles have a ratio of the amphiphilic block copolymer to the therapeutic protein of about 5.

In certain aspects, the concentration of the polyelectrolyte complex is about 2 mg/mL. In certain aspects, the concentration of the amphiphilic block copolymer is about 10 mg/mL.

In certain aspects, the one or more nanoparticles have an average hydrodynamic size of between about 20 nm and about 200 nm. In certain aspects, the one or more nanoparticles have a polydispersity index of between about 0.05 to about 0.5. In certain aspects, the one or more nanoparticles have an average zeta potential of between about −5 mV to −40 mV. In certain aspects, the one or more nanoparticles have an average zeta potential of −20.8±2.6 mV.

In certain aspects, the one or more nanoparticles have an encapsulation efficiency of therapeutic small protein in the amphiphilic block copolymer is between about 60% to about 99%. In certain aspects, the one or more nanoparticles have an average loading level of the therapeutic protein in the amphiphilic block copolymer is between about 0.1 to about 50% by weight. In certain aspects, the one or more nanoparticles have an average loading level of the therapeutic small protein in the amphiphilic block copolymer is about 14.2±0.9%.

In certain aspects, the volume ratio of the water-miscible organic solvent to water in the aqueous solution, i.e., the organic solvent/water ratio, in the FNP process is between about 0.1 to 2. The organic solvent/water ratio is controlled by adjusting the flow rate ratio of organic solvent jet to the aqueous solution jet. In certain aspects, the organic solvent/water ratio in the FNP process is about 1.

In some aspects, the presently disclosed subject matter provides a nanoparticle comprising an amphiphilic block copolymer having a polyelectrolyte complex comprising one or more therapeutic small proteins and a counter ion polymer encapsulated therein, wherein the counter ion polymer has a charge enabling it to bind electrostatically to the one or more therapeutic small proteins.

In certain aspects, the therapeutic small protein comprises a growth factor or a proteoglycan. In particular aspects, the therapeutic small protein comprises insulin-like growth factor 1 (IGF-1) or agrin.

In certain aspects, the counter ion polymer is selected from dextran sulfate (DS), heparin (heparin sulfate), hyaluronic acid, and combinations thereof. In particular aspects, the counter ion comprises dextran sulfate.

In certain aspects, the amphiphilic block copolymer is selected from poly(ethylene glycol)-block-poly(D,L-lactic acid) (PEG-b-PDLLA), poly(ethylene glycol)-block-poly(ε-caprolactone) (PEG-b-PCL), poly(ethylene glycol)-block-poly(lactic-co-glycolic acid) (PEG-b-PLGA) and combinations thereof. In particular aspects, the amphiphilic block copolymer comprises poly(ethylene glycol)-block-poly(ε-caprolactone) (PEG-b-PCL). In more particular aspects, the amphiphilic block copolymer comprises PEG10k-b-PCL40k.

In particular aspects, the therapeutic small protein comprises IGF-1, the counter ion polymer comprises dextran sulfate, and the amphiphilic block polymer comprises poly(ethylene glycol)-block-poly(ε-caprolactone) (PEG-b-PCL).

In certain aspects, the nanoparticle has a loading level of therapeutic small protein between about 0.1% and 50% by weight.

In particular aspects, the nanoparticle is biodegradable.

In other aspects, the presently disclosed subject matter provides a composition comprising a presently disclosed nanoparticle and a hydrogel, wherein the nanoparticle is distributed throughout the hydrogel. In certain aspects, the hydrogel comprises a fibrin gel or a nanofiber-hyaluronic acid hydrogel composite (NHC).

In other aspects, the presently disclosed subject matter provides a method for treating peripheral nerve injury, the method comprising administering a presently disclosed nanoparticle or a composition thereof to a subject in need of treatment thereof.

In certain aspects, the method comprises administering the nanoparticle or composition to one or more of a denervated muscle, an injured nerve of the subject. In particular aspects, the nanoparticle or composition thereof is administered to at least one of the denervated muscle, the injured nerve, near the injured nerve during or after surgical repair of the denervated muscle or injured nerve.

In certain aspects, the administration comprises a controlled release of the small therapeutic protein from the nanoparticle or composition.

In certain aspects, the method further comprises interval re-dosing the subject with the nanoparticle or composition thereof. In particular aspects, the interval re-dosing is conducted under ultrasound guidance.

In certain aspects, the subject after being administered the nanoparticle or composition thereof exhibits an improved motor recovery. In particular aspects, the motor recovery is evidenced through neuromuscular reinnervation, nerve regeneration, a decrease in Schwann cell (SC) senescence, axonal regrowth, an amelioration of denervation-induced muscle atrophy, and combinations thereof. In particular aspects, the amelioration of denervation-induced muscle atrophy is evidenced by an increase in mean myofiber cross-sectional area. In particular aspects, the improved motor recovery is evidenced by an increase in Schwann cell proliferation. In yet more particular aspects, the improved motor recovery is evidenced by an increase in grip strength.

Certain aspects of the presently disclosed subject matter having been stated hereinabove, which are addressed in whole or in part by the presently disclosed subject matter, other aspects will become evident as the description proceeds when taken in connection with the accompanying Examples and Figures as best described herein below.

### BRIEF DESCRIPTION OF THE DRAWINGS

The patent or application file contains at least one drawing executed in color. Copies of this patent or patent application publication with color drawings will be provided by the Office upon request and payment of the necessary fee.

Having thus described the presently disclosed subject matter in general terms, reference will now be made to the accompanying drawings, which are not necessarily drawn to scale, and wherein:

FIG. 1 illustrates representative procedures for (Step 1) IGF-1/DS PEC assembly and (Step 2) encapsulation of IGF-1/DS PEC in PEG-b-polyester NPs via flash nanoprecipitation (FNP) method using a 4-inlet multi-inlet vortex mixer:

FIG. 2A, FIG. 2B, and FIG. 2C illustrate: (FIG. 2A) Study design schematic. In Study 1, the median and ulnar nerves in the IGF-1 treated experimental group and untreated negative control group were transected and left unrepaired. Eight weeks later, nerve and muscle tissue were analyzed to evaluate the treatment effect of IGF-1 on chronically-denervated muscle and Schwann cells in the absence of subsequent regeneration. Not shown: positive controls were harvested one week following nerve transection, correlating with maximal Schwann cell proliferation. In Study 2, the median nerve in the experimental and negative control groups was cut and left in discontinuity for eight weeks followed by nerve repair (via ulnar-to-median nerve transfer) to evaluate nerve regeneration, muscle reinnervation and functional recovery in the setting of induced chronic denervation: weekly evaluation of functional recovery was then performed until sacrifice at 15 weeks. Not shown: the positive control group underwent nerve transection and immediate repair (ulnar-to-median nerve transfer) without being subjected to a period of chronic denervation prior to repair: (FIG. 2B) Illustration of median nerve transection with administration of NPs around the distal nerve and into the denervated muscle compartment (first surgery of study 2): (FIG. 2C) Depiction of second surgery of study 2, with ulnar-to-median nerve transfer and NP re-administration:

FIG. 3A, FIG. 3B, FIG. 3C, FIG. 3D, FIG. 3E, FIG. 3F, and FIG. 3G demonstrate that: (FIG. 3A) NP size was smallest and most homogenous at a pH of 4. (FIG. 3B) Complexation efficiency is optimized with increased IGF-1 concentrations; and a DS/IGF-1 mass ratio of 0.2 achieves the highest complexation efficiency. (FIG. 3C) The hydrodynamic size of NP decreases with the flow rate; and the PDI of NP decreases as the flow rate increases from 1 mL/min to 6 mL/min. The highest encapsulation efficiency is achieved at the flow rate of 5 mL/min. (FIG. 3D) The size uniformity of NP is improved with increasing flow rate. The three traces in each graph indicate three separate runs of the NP preparation. Scale bar in TEM: 200 nm. (FIG. 3E) Comparison of amphiphilic block copolymers demonstrating optimal encapsulation efficiency with PEG-PCL. (FIG. 3F) The encapsulation efficiency increases with the increasing PEG-PCL: IGF-1 mass ratio. (FIG. 3G) All 3 block copolymers produce uniform IGF-1/DS encapsulated NPs. The two traces in each graph indicate two separate runs of the NP preparation for each polymer:

FIG. 4 shows the size distribution of NPs, as measured by dynamic light scattering, become more uniform as the mixing flow rate is increased. The three traces in each graph indicate three separate runs of the NP preparation:

FIG. 5 shows the screening of water-miscible solvents in the FNP process. The size uniformity of NP is improved when ACN or DMF is applied to dissolve the polyesters. The selection of DMSO and THF produce NPs with multiple peaks and broader size distribution:

FIG. 6 shows screening of the organic solvent/water ratio in the FNP process. The size uniformity of NP is improved when a higher organic solvent/water ratio (>1) was applied. The organic solvent/water ratio of I was selected for the final formulation:

FIG. 7A, FIG. 7B, FIG. 7C, FIG. 7D, FIG. 7E, FIG. 7F, and FIG. 7G show: (FIG. 7A) DLS and TEM characterization of the optimized formulation used for in vitro and in vivo experiments. Scale bar in inset: 100 nm. (FIG. 7B) The elemental distribution of the IGF-1 loaded NPs. IGF-1 (indicated by the nitrogen signal) distributed throughout the NP along with DS (indicated by the sulfur signal), corroborated with the hypothesis that IGF-1 and DS were encapsulated inside NP in a complex form. As the IGF-1 releases from the NP, the N signal decreases with time, whereas the S signal remains inside the NP, indicating slower release kinetics of DS (FIG. 7C). The percentage of cumulative IGF-1 release profiles from IGF-1-encapsulated NPs in PBS or in fibrin gel, IGF-1/DS PECs in PBS or in fibrin gel, as compared with IGF-1 loaded in fibrin gel. IGF-1 encapsulated NPs when loaded in a fibrin gel gives a slightly delayed release profile. (FIG. 7D. FIG. 7E, FIG. 7F, FIG. 7G). Bioactivity of released IGF-1 was evaluated by measuring the mitogenic activity in human Schwann cells (FIG. 7D, FIG. 7E) and c2c12 myocytes (FIG. 7F, FIG. 7G):

FIG. 8A, FIG. 8B, FIG. 8C, FIG. 8D, and FIG. 8E demonstrate IGF-1 NPs prevent effects of chronic denervation on myofibers and Schwann cells. (FIG. 8A) Representative images of laminin-stained muscle cross-sections that were denervated for 8 weeks in negative control and experimental groups and naïve (non-denervated) in the positive control group. Scale bar: 50 μm. (FIG. 8B) The IGF-1 treated experimental groups demonstrated greater mean myofiber cross sectional area (CSA) (less muscle atrophy) than the untreated negative control group: mean CSA was greatest in the positive control group. (FIG. 8C) Representative staining of median nerve cross-sections for proliferating (Ki67) Schwann cells (S100) taken distal to the site of nerve transection following one week of denervation in the positive control group to induce maximal proliferation and following 8 weeks of denervation in the experimental and negative control groups to induce chronic denervation. Scale bar: 10 μm. (FIG. 8D) The percentage of Schwann cells that were proliferating was greater in the experimental group than the negative control group, with no difference seen between the experimental and positive control groups. (FIG. 8E) ErbB3 expression, a marker of Schwann cell (SC) proliferation, was up-regulated 19-fold in the experimental cohort relative to negative controls:

FIG. 9A, FIG. 9B, FIG. 9C, FIG. 9D, FIG. 9E, and FIG. 9F demonstrate that IGF-1 NP improves histological and functional recovery after chronic nerve repair. (FIG. 9A) Representative staining of neuromuscular junctions on longitudinal muscle sections 15 weeks following nerve repair. Scale bar: 100 μm. (FIG. 9B) The IGF-1 treated experimental group demonstrated greater percent reinnervation of neuromuscular junctions than the untreated negative control group. (FIG. 9C) Representative images showing laminin staining of muscle cross-sections at 15 weeks following nerve repair. Scale bar: 50 μm. (FIG. 9D) The IGF-1-treated experimental group demonstrated greater myofiber cross-sectional area (less muscle atrophy) than the untreated negative control group, and similar myofiber cross-sectional area to the positive control group that was not subjected to chronic denervation prior to nerve repair. (FIG. 9E) Illustration demonstrating performance of stimulated grip strength testing (SGST): with the animal anesthetized, the median nerve is percutaneously stimulated to induce maximal tetanic contraction of the digital flexors and grasp of the handlebar: the force transducer is then distracted away from the animal until grasp is lost and load to failure is measured. (FIG. 9F) Grip strength was significantly greater in the IGF-1-treated experimental group compared to the negative control group beginning at week 11 until sacrifice at 15 weeks; no differences in grip strength were observed between the experimental and positive control groups:

FIG. 10A, FIG. 10B, and FIG. 10C show in vivo live imaging of NP biodistribution after injection around the median nerve. NPs were conjugated to fluorescent Cy7.5. Fluorescent intensity demonstrates prolonged local retention but decreases with time (FIG. 10A). The quantification of NP signal in vivo for the first 7 days: NPs in fibrin were retained longer than NPs suspended in PBS (FIG. 10B). There was no accumulation of signal in other major organs, but was present in muscle at 28 days (FIG. 10C);

FIG. 11A, FIG. 11B, and FIG. 11C show the IGF-1 concentrations measured with ELISA in treated muscle and nerve tissue, as well as serum. The tissue concentration of IGF-1 was markedly higher in the experimental cohort (FIG. 11A). Serum IGF-1 concentration increased in the experimental group throughout the study (FIG. 11B), but systemic distribution remained low relative to tissue concentrations (FIG. 11B and FIG. 11C);

FIG. 12 shows the composition of the drug carrier-nanofiber/hydrogel composite:

FIG. 13A, FIG. 13B, FIG. 13C, FIG. 13D, FIG. 13E, FIG. 13F, and FIG. 13G show the characterization of NHC in terms of morphology and pro-regenerative responses:

FIG. 14 demonstrates the mixing of IGF-1 NP with NHC to loaded IGF-1 NP;

FIG. 15 demonstrates the release kinetics of IGF-1 NP in NHC:

FIG. 16 shows the confocal fluorescence images of IGF-1, NP and NHC in vivo at different time points:

FIG. 17 demonstrates quantification of NP signal retention in vivo in the muscle when delivered in PBS or NHC:

FIG. 18 shows weight loss of NHC over time in comparison with IGF-1 retention inside the NHC in vivo;

FIG. 19 shows IGF-1 in vivo concentrations with every 6-week injection in rats.

Injections were conducted at time points with red arrows:

FIG. 20 demonstrates functional recovery to show in vivo efficacy of IGF-1 NP/NHC in rats. Injections were conducted at time points with red arrows:

FIG. 21 shows IGF-1 in vivo concentrations with every 6-week injection in macaques. Injections were conducted at time points with red arrows:

FIG. 22 shows the experimental apparatus of ultrasound-guided macaque grip strength measurements:

FIG. 23 demonstrates functional recovery to show in vivo efficacy of IGF-1 NP/NHC in macaques. Injections were conducted at time points with red arrows;

FIG. 24A and FIG. 24B show a schematic of agrin NP production:

FIG. 25 shows the hydrodynamic size of agrin NP measured by DLS:

FIG. 26 demonstrates long-term (>100 days) release of agrin from NP:

FIG. 27 demonstrates that agrin-NP treatment preserved pretzel-like neuromuscular junction morphology and reduced degradation as compared to empty-NP and free agrin treated animals (p<0.001):

FIG. 28 shows agrin in vivo concentrations of different doses and formulations; and

FIG. 29 shows that animals undergoing chronic denervation with agrin-NP/NHC therapy (green) demonstrated greater grip strength recovery compared to free agrin (blue) and empty-NP/NHC (red) treated animals.

### DETAILED DESCRIPTION

The presently disclosed subject matter now will be described more fully hereinafter with reference to the accompanying drawings, in which some, but not all embodiments of the presently disclosed subject matter are shown. Like numbers refer to like elements throughout. The presently disclosed subject matter may be embodied in many different forms and should not be construed as limited to the embodiments set forth herein: rather, these embodiments are provided so that this disclosure will satisfy applicable legal requirements. Indeed, many modifications and other embodiments of the presently disclosed subject matter set forth herein will come to mind to one skilled in the art to which the presently disclosed subject matter pertains having the benefit of the teachings presented in the foregoing descriptions and the associated drawings. Therefore, it is to be understood that the presently disclosed subject matter is not to be limited to the specific embodiments disclosed and that modifications and other embodiments are intended to be included within the scope of the appended claims.

### A. Method for Preparing a Nanoparticle

In some embodiments, the presently disclosed subject matter provides a method for preparing a nanoparticle comprising an amphiphilic block copolymer having a polyelectrolyte complex comprising one or more therapeutic small proteins and a counter ion polymer encapsulated therein, the method comprising: (a) assembling the polyelectrolyte complex comprising a therapeutic small protein and a counterion polymer, wherein the counter ion polymer has a charge enabling it to bind electrostatically to the one or more therapeutic small proteins; and (b) encapsulating the polyelectrolyte complex with an amphiphilic block copolymer through a flash nanoprecipitation (FNP) process.

As used herein, the term “polyelectrolyte complexes” (also known as polyelectrolyte coacervates or “PECs”) are the association complexes formed between oppositely charged particles (e.g., polymer-polymer, polymer-drug, and polymer-drug-polymer). Polyelectrolyte complexes are formed due to electrostatic interaction between oppositely charged polyions, i.e., water-soluble polycations and water-soluble polyanions. See U.S. Pat. No. 10,441,549 to Mao et al., for “Methods of preparing polyelectrolyte complex nanoparticles,” issued Oct. 15, 2019, which is incorporated herein in its entirety.

As used herein, the term “water-soluble” refers to the ability of a compound to be able to be dissolved in water. As used herein, the terms “continuous” or “continuously” refer to a process that is uninterrupted in time, such as the generation of PEC nanoparticles while at least two presently disclosed streams are flowing into a confined chamber.

Flash nanoprecipitation (FNP) offers a continuous and scalable process that has been used for the production of block copolymer nanoparticles. Flash nanoprecipitation (FNP) uses a kinetic controlled process to generate nanoparticles in a continuous and scalable manner by using confined impinging jet (CIJ) or multi-inlet vortex mixer (MIVM) device. The rapid micromixing conditions of FNP (on the order of 1 msec) establishes homogeneous supersaturation conditions and controlled precipitation of hydrophobic solutes (organic or inorganic) using block copolymer self-assembly. Compared to bulk preparation methods, the FNP process allows for the formation of uniform aggregates with tunable size in a continuous flow operation process, which is amenable for scale-up production. This process also offers a higher degree of versatility and control over particle size and distribution, higher drug encapsulation efficiency, and improved colloidal stability.

In some embodiments, the forming of the nanoparticles occurs by the precipitation of the biodegradable polymer together with the polyelectrolyte complex (PEC).

A two-step process for forming PEC-containing nanoparticles is provided in International PCT Patent Application Publication No. WO/2019/148147, to Mao et al., for “Polymeric nanoparticle compositions for encapsulation and sustained release of protein therapeutics, published Aug. 1, 2019). In this process, the polycation solution (i.e., the solution comprising the one or more therapeutic small proteins), a polyanion solution (i.e., one or more counter ion polymers, e.g., dextran sulfate, heparin sulfate, and the like), and block copolymer dissolved in a water miscible solvent are introduced into a defined chamber at an optimized set of flow rates to achieve efficient mixing, therefore obtaining nanoparticles with efficient loading of the one or more therapeutic small proteins.

In contrast to processes known in the art, the presently disclosed method includes a pre-formulation step to assemble the polyelectrolyte complex comprising a therapeutic small protein and a counterion polymer prior to encapsulating the polyelectrolyte complex with the amphiphilic block copolymer via FNP.

In particular embodiments, the flash nanoprecipitation (FNP) method comprises a multi-inlet vortex mixer. In certain embodiments, the multi-inlet vortex mixer comprises four inlets.

In particular embodiments, one inlet of the multi-inlet vortex mixer is in fluid communication with a source of the polyelectrolyte complex, one inlet of the multi-inlet vortex mixer is in fluid communication with a source of the amphiphilic block copolymer, and two inlets of the multi-inlet vortex mixer are in fluid communication with a source of water.

In such embodiments, the polyelectrolyte complex solution and the amphiphilic block copolymer solution are introduced simultaneously into the vortex mixer along with water.

In certain embodiments, the assembling of the polyelectrolyte complex is conducted at a pH that is below the isoelectric point of the protein, under which condition the therapeutic protein carries net positive charges.

In certain embodiments, the assembling of the polyelectrolyte complex is conducted at a pH from about 1 to about 5, including 1, 1.5, 2, 2.5, 3, 3.5, 4, 4.5, and 5. In particular embodiments, the pH is about 4.

In some embodiments, the flow rate of each jet of the vortex mixer as a range from about 1 mL/min to about 60 mL/min, including 1, 1.5, 2, 2.5, 3, 3.5, 4, 4.5, 5, 5.5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, and 60 mL/min. In particular embodiments, the flow rate is between about 1 to about 10 mL/min, including about 1, 2, 3, 4, 5, 6, 7, 8, 9, and 10 mL/min.

In some embodiments, the assembling of the polyelectrolyte complex is conducted at a pH having a range from about 2 to about 5, including 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9 and 5.0. In particular embodiments, the assembling of the polyelectrolyte complex is conducted at a pH of about 4.

In certain embodiments, a hydrodynamic size of the nanoparticle decreases with flow rate.

In certain embodiments, a PDI of the nanoparticles decreases as the flow rate increases from 1 mL/min to 60 mL/min. In particular embodiments, each jet has a flow rate of about 5 mL/min.

In certain embodiments, a size uniformity of the nanoparticle improves with increasing flow rate.

In certain embodiments, the volume ratio of the water-miscible organic solvent to water in the aqueous solution, i.e., the organic solvent/water ratio, in the FNP process is between about 0.1 to 2. The organic solvent/water ratio is controlled by adjusting the flow rate ratio of organic solvent jet to the aqueous solution jet. In certain embodiments, the organic solvent/water ratio in the FNP process is about 1.

In certain embodiments, an encapsulation efficiency increases with increasing amphiphilic block polymer:therapeutic small protein, e.g., PEG-PCL:IGF-1 mass ratio.

The amount of unencapsulated protein was measured by the BCA assay, and the encapsulation efficiency (EE) was calculated using the following formula:

$$EE ( \% ) = ( {m}_{total} - {m}_{free} ) / {m}_{total} \times 100 \% ,$$

where mₜₒₜₐₗ represents the mass of the total feeding protein and m_{free} represents the mass of free protein in the supernatant.

In some embodiments, the nanoparticles have an encapsulation efficiency of between about 50% to about 100%, including 50, 55, 60, 65, 70, 75, 80, 85, 86, 87, 88, 89, 90, 91, 92, 93, 94, 95, 96, 97, 98, 99, 99.5, 99.9, and 100%. In certain embodiments, the encapsulation efficiency is between about 50% and 99%. In certain embodiments, the encapsulation efficiency is between about 60% and 99%. In more certain embodiments, the encapsulation efficiency is between about 80% and 99%. In even more certain embodiments, the encapsulation efficiency is between about 85% and 99%. In particular embodiments, the nanoparticle has an encapsulation efficiency of therapeutic small protein in the amphiphilic block copolymer of about 88.4±3.9%.

In some embodiments, the polyelectrolyte complex has a mass ratio of counter ion polymer to therapeutic small protein from about 0.1 to about 10, including about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 3.0, 4.0, 5.0, 6.0, 7.0, 8.0, 9.0, and 10.0. In certain embodiments, the polyelectrolyte complex comprises a mass ratio of counter ion polymer to therapeutic small protein, e.g., DS/IGF-1, of about 0.2.

In certain embodiments, the polyelectrolyte complex suspended in a DMSO solution.

In some embodiments, the water-miscible organic solvent is selected from the group consisting of acetonitrile (ACN), dimethyl sulfoxide (DMSO), tetrahydrofuran (THF), dimethylformamide (DMF), ethanol, isopropyl alcohol (IPA), hexafluoroisopropanol (HFIP), and combinations thereof. In certain embodiments, the water-miscible organic solvent is acetonitrile or dimethylformamide. In particular embodiments, the water-miscible organic solvent is acetonitrile. In other embodiments, the water-miscible organic solvent is dimethylformamide.

In certain embodiments, the method comprises a ratio of the amphiphilic block copolymer to the small therapeutic protein of about 5.

In certain embodiments, the concentration of the polyelectrolyte complex is about 2 mg/mL.

In certain embodiments, the concentration of the amphiphilic block copolymer is about 10 mg/mL.

In some embodiments, the nanoparticles range in size from about 20 nm to about 1000 nm in diameter, including about 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, 430, 435, 440, 445, 450, 455, 460, 465, 470, 475, 480, 485, 490, 495, 500, 505, 510, 515, 520, 525, 530, 535, 540, 545, 550, 555, 560, 565, 570, 575, 580, 585, 590, 600, 605, 610, 615, 620, 625, 630, 635, 640, 645, 650, 655, 660, 665, 670, 675, 680, 685, 690, 695, 700, 705, 710, 715, 720, 725, 730, 735, 740, 745, 750, 755, 760, 770, 775, 780, 785, 790, 795, 800, 805, 810, 815, 820, 825, 830, 835, 840, 845, 850, 855, 860, 865, 870, 875, 880, 885, 890, 895, 900, 905, 910, 915, 920, 925, 930, 935, 940, 945, 950, 955, 960, 965, 970, 975, 980, 985, 990, 995, and 1000 nm. For example, in some embodiments, the present nanoparticles have an average particle size of less than about 1000 nm, less than about 900, less than about 800 nm, less than about 700 nm, less than about 600 nm, less than about 500 nm, less than about 400 nm, less than about 300 nm, less than about 200 nm, and less than about 100 nm. In certain embodiments, the nanoparticle has a size ranging from about 50 nm to about 500 nm. In more certain embodiments, the nanoparticle has a size of about 50 nm. In particular embodiments, the nanoparticle has an intensity-average hydrodynamic size of 53.4±1.4 nm.

In some embodiments, the nanoparticles have a polydispersity index (PDI) ranging from about 0.05 to about 0.5, including a PDI of about 0.05, 0.06, 0.07, 0.08, 0.09, 0.1, 0.11, 0.12, 0.13, 0.14, 0.15, 0.16, 0.17, 0.18, 0.19, 0.20, 0.21, 0.22, 0.23, 0.24, 0.25, 0.26, 0.27, 0.28, 0.29, 0.30, 0.40, and 0.50. In certain embodiments, the nanoparticles have a PDI ranging from about 0.1 to about 0.5. In more certain embodiments, the nanoparticles have a PDI ranging from about 0.1 to about 0.2. In particular embodiments, the nanoparticles have a PDI of about 0.13±0.02.

In certain embodiments, the nanoparticle has an average zeta potential of between about −5 mV to about −40 mV, including about −5, −6, −7, −8, −9, −10, −11, −12, −13, −14, −15, −16, −17, −18, −19, −20, −21, −22, −23, −24, −25, −26, −27, −28, −29, −30, −31, −32, −33, −34, −35, −36, −37, −38, −39, and −40 mV. In particular embodiments, the average zeta potential is about −20.8±2.6 mV.

In certain embodiments, the nanoparticle comprises an average loading level of the therapeutic small protein in the amphiphilic block copolymer is about 0.1 to about 50%, including about 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and 50%. In particular embodiments, the average loading level is between about 10% to about 14%. In more particular embodiments, the average loading level is about 14.2±0.9%.

### B. Nanoparticles

In some embodiments, the presently disclosed subject matter provides a nanoparticle comprising an amphiphilic block copolymer having a polyelectrolyte complex comprising one or more therapeutic small proteins and a counter ion polymer encapsulated therein, wherein the counter ion polymer has a charge enabling it to bind electrostatically to the one or more therapeutic small proteins.

As used herein, the term “amphiphilic block copolymer includes at least one polymer having a hydrophilic and a hydrophobic component.

In certain embodiments, the amphiphilic block copolymer is selected from poly(ethylene glycol)-block-poly(D,L-lactic acid) (PEG-b-PDLLA), poly(ethylene glycol)-block-poly(ε-caprolactone) (PEG-b-PCL), poly(ethylene glycol)-block-poly(lactic-co-glycolic acid) (PEG-b-PLGA) and combinations thereof.

In particular embodiments, the amphiphilic block copolymer comprises poly(ethylene glycol)-block-poly(ε-caprolactone) (PEG-b-PCL). In certain embodiments, the PEG-b-PCL copolymer comprises a PEGₓ-b-PCL_{y} copolymer, wherein x is a molecular weight of PEG having a range from about 350 to 10,000 (10k), including 350, 500, 1k, 2k, 5k, and 10k) and y is a molecular weight of PCL having a range from about 1k to about 40 k, including 1k, 2k, 5k, 10k, 20k, and 40k. In particular embodiments, the amphiphilic block copolymer comprises PEG10k-b-PCL40k.

Therapeutic proteins can be used to replace a protein that is abnormal or deficient in a particular disease. They can also augment the body's supply of a beneficial protein to help reduce the impact of disease. Therapeutic proteins include antibody-based drugs, Fc fusion proteins, anticoagulants, blood factors, bone morphogenetic proteins, engineered protein scaffolds, enzymes, growth factors, hormones, interferons, interleukins, and thrombolytics.

The terms “polypeptide” and “protein” as used herein refer to a polymer of amino acids. As used herein, a “peptide” refers to short chain of amino acid monomers, such as about 50 or fewer amino acids.

Proteins generally contain from 40 to 1000 amino acid residues (AAs) per polypeptide chain. Polypeptides containing about 100 AAs or less are considered to be small proteins (SPs). In some embodiments, a small protein can comprise about 100 amino acid residues, in some embodiments, 100, 99, 98, 97, 96, 95, 94, 93, 92, 91, 90, 89, 88, 87, 86, 85, 84, 83, 82, 81, 80, 79, 78, 77, 76, 75, 74, 73, 72, 71, 70, 69, 68, 67, 66, 65, 64, 63, 62, 61, 60, 59, 58, 57, 56, 55, 54, 53, 52, 51, 50, 49, 48, 47, 46, 45, 44, 43, 42, 41, and 40 AA.

In some embodiments, the therapeutic small protein comprises between about 90 and about 100 AAs, in some embodiments between about 80 and about 90 AAs, in some embodiments, between about 70 and 80 AAs, in some embodiments, between about 60 and about 70 AAs, in some embodiments, between about 50 and about 60 AAs, between about 40 and about 50 AAs, between about 30 and about 40 AAs, between about 20 and 30 AAs, between about 10 and about 20 AAs, and between about 1 and about 10 AAs.

In some embodiments, the therapeutic protein comprises a growth factor. As used herein, a “growth factor” refers to a substance, such as a protein or hormone, which is capable of stimulating cellular growth, proliferation, healing, and/or cellular differentiation. Non-limiting examples of growth factors include platelet derived growth factor (PDGF), transforming growth factor beta. (TGF-beta), insulin-like growth factor-I (IGF-I), insulin-like growth factor-II (IGF-II), fibroblast growth factor (FGF), beta-2-microglobulin (BDGF II), and bone morphogenetic factors.

In particular embodiments, the growth factor comprises insulin-like growth factor 1 (IGF-1) or agrin. IGF-1 is a protein that in humans is encoded by the IGF1 gene. IGF-1 consists of 70 amino acids in a single chain with three intramolecular disulfide bridges and has a molecular weight of 7,649 Daltons.

In some embodiments, the therapeutic small protein comprises a recombinant form of human insulin-like growth factor 1. In certain embodiments, the recombinant form of human insulin-like growth factor 1 comprises recombinant human insulin-like growth factor-1 (rhIGF-1) (also known as mecasermin, which is marketed under the brand name Increlex® by Ipsen Biopharmaceuticals, Inc.)

As used herein, the term “counter ion polymer” includes a polymer having a charge so that the polymer is able to bind electrostatically to the one or more neuromodulators. Examples include a protein that is net positively charged the binds to a counter ion polymer that has a net negative charge or vice versa.

In certain embodiments, the counter ion polymer is negatively charged. In some embodiments, the counter ion polymer is selected from dextran sulfate (DS), heparin (heparin sulfate), hyaluronic acid, and combinations thereof. In particular embodiments, the counter ion comprises dextran sulfate.

Other anionic polymers suitable for use with the presently disclosed compositions and methods include, but are not limited to, poly(aspartic acid), poly(glutamic acid), negatively charged block copolymers, alginate, tripolyphosphate (TPP), and oligo (glutamic acid).

In more particular embodiments, the therapeutic small protein comprises IGF-1, the counter ion polymer comprises dextran sulfate, and the amphiphilic block polymer comprises poly(ethylene glycol)-block-poly(ε-caprolactone) (PEG-b-PCL).

In certain embodiments, the nanoparticle is biodegradable. Generally, as used herein, the term “biodegradable” refers to a nanoparticle, or a polymeric component of the nanoparticle, which is degraded in a physiological environment through an enzymatic process or through non-enzymatic hydrolysis. Thus, as used herein, a biodegradable nanoparticle degrades over a period of time in vivo to soluble products that can be easily removed from an implant site and/or excreted from the subject's body.

In some embodiments, the biodegradable polymer is a copolymer selected from the group consisting of poly(L-lactic acid) (PLLA), polyglycolic acid (PGA), poly(D,L-lactic-co-glycolic acid) (PLGA), polycaprolactone (PCL), their PEGylated block copolymers, and combinations thereof. In certain aspects, the biodegradable polymer is selected from the group consisting of polyethylene glycol (PEG)-b-PLLA, PEG-b-PLGA, PEG-b-PCL, and combinations thereof.

### C. Compositions

In some embodiments, the presently disclosed subject matter provides a composition comprising a presently disclosed nanoparticle and a crosslinked hydrophilic polymer, wherein the nanoparticle is distributed throughout the crosslinked hydrophilic polymer.

In certain embodiments, the crosslinked hydrophilic polymer comprises a hydrogel. In certain aspects, the hydrogel comprises a natural or synthetic hydrophilic polymer selected from the group consisting of hyaluronic acid, chitosan, heparin, alginate, fibrin, polyvinyl alcohol, polyethylene glycol, sodium polyacrylate, an acrylate polymer, and copolymers thereof. In particular embodiments, the hydrogel comprises a fibrin gel or a nanofiber-hyaluronic acid hydrogel composite (NHC).

### D. Methods for Treating a Peripheral Nerve Injury

In some embodiments, the presently disclosed subject matter provides a method for treating a peripheral nerve injury, the method comprising administering a presently disclosed nanoparticle or a presently disclosed composition to a subject in need of treatment thereof.

A peripheral nerve fiber contains an axon (or long dendrite), myelin sheath, their Schwann cells, and the endoneurium. Peripheral nerve injury can be classified as: neurapraxia, in which the nerve remains intact but signaling ability is damaged; axonotmesis, in which the axon is damaged but the surrounding connecting tissue remains intact; and neurotmesis, in which both the axon and connective tissue are damaged.

More particularly, neurapraxia is a temporary interruption of conduction without loss of axonal continuity. In neurapraxia, there is a physiologic block of nerve conduction in the affected axons. The endoneurium, perineurium, and the epineurium are intact and there is no Wallerian degeneration. Conduction is intact in the distal segment and proximal segment, but no conduction occurs across the area of injury.

Axonotmesis involves loss of the relative continuity of the axon and its covering of myelin, but preservation of the connective tissue framework of the nerve (the encapsulating tissue, the epineurium and perineurium, are preserved). Wallerian degeneration occurs distal to the site of injury and there are sensory and motor deficits distal to the site of lesion and there is no nerve conduction distal to the site of injury (up to 3 to 4 days after injury).

Neurotmesis is a total severance or disruption of an entire nerve fiber. Neurotmesis may be partial or complete. Wallerian degeneration occurs distal to the site of injury. In neurotmesis, sensory-motor problems and autonomic function defect are severe. There is no nerve conduction distal to the site of injury (3 to 4 days after lesion). Surgical intervention typically is necessary.

In certain embodiments, the method comprises administering the nanoparticle or composition within, overlying or in close proximity to a denervated muscle, along the epineurium or in close proximity to an injured nerve tissue, near the injured nerve tissue, and combinations thereof, of the subject. In some embodiments, the administration of the nanoparticle or composition within during surgical repair of the denervated muscle or injured nerve.

As used herein, the term “denervation” relates to a loss of axons or fibers within a nerve and/or supplying innervation to muscle and/or skin. Causes of denervation include, but are not limited to disease, chemical toxicity, physical injury, or intentional or unintentional surgical trauma to a nerve.

In certain embodiments, the administering comprises a controlled release of the small therapeutic protein comprising the nanoparticle or composition.

In particular embodiments, the method further comprises interval re-dosing the subject with the nanoparticle or composition. In certain embodiments, interval re-dosing is performed percutaneously after nanoparticle payloads have been completely released based on the need of treatment.

The interval redosing schedule will vary depending on the specific release rate for the nanoparticle encapsulated small protein therapeutic formulation. A representative re-dosing schedule is about every 6 weeks, but, in some embodiments, could be about every one week, about every two weeks, about every three weeks, about every four weeks, about every five weeks, about every six weeks, about every seven weeks, about every eight weeks, about every nine weeks, and about every ten weeks.

Re-dosing involves percutaneous injection into, overlying, or in close proximity to denervated muscle groups and along the epineurium or in close proximity to the affected nerve tissue distal to the site of injury.

In more particular embodiments, the interval re-dosing is conducted under ultrasound guidance.

In certain embodiments, the subject after being administered the nanoparticle or composition exhibits an improved motor recovery.

In some embodiments, the presently disclosed method ameliorates one or more effects of denervation, including chronic denervation, on myofibers and Schwann cells. For example, the mean myofiber cross-sectional area of myofibers in a subject undergoing treatment with the presently disclosed nanoparticles increases, which is an indication of less muscle atrophy due to chronic denervation. Further, Schwann cell proliferation increases, compared to a control, for subjects treated with the presently disclosed nanoparticles.

In particular embodiments, the motor recovery improves through one or more of neuromuscular reinnervation, e.g., an increase in reinnervation of neuromuscular junctions, nerve regeneration, a decrease in SC senescence, axonal growth or regeneration, an amelioration of denervation-induced muscle atrophy, and combinations thereof.

The “subject” treated by the presently disclosed methods in their many embodiments is desirably a human subject, although it is to be understood that the methods described herein are effective with respect to all vertebrate species, which are intended to be included in the term “subject.” Accordingly, a “subject” can include a human subject for medical purposes, such as for the treatment of an existing condition or disease or the prophylactic treatment for preventing the onset of a condition or disease, or an animal subject for medical, veterinary purposes, or developmental purposes. Suitable animal subjects include mammals including, but not limited to, primates, e.g., humans, monkeys, apes, and the like: bovines, e.g., cattle, oxen, and the like: ovines, e.g., sheep and the like: caprines, e.g., goats and the like: porcines, e.g., pigs, hogs, and the like: equines, e.g., horses, donkeys, zebras, and the like: felines, including wild and domestic cats: canines, including dogs: lagomorphs, including rabbits, hares, and the like; and rodents, including mice, rats, and the like. An animal may be a transgenic animal. In some embodiments, the subject is a human including, but not limited to, fetal, neonatal, infant, juvenile, and adult subjects. Further, a “subject” can include a patient afflicted with or suspected of being afflicted with a condition or disease. Thus, the terms “subject” and “patient” are used interchangeably herein. The term “subject” also refers to an organism, tissue, cell, or collection of cells from a subject.

As used herein, the terms “treating” or “treatment” means to prevent, reduce the occurrence, alleviate, or to eliminate an undesirable condition, either temporarily or permanently.

As used herein, the term “alleviating” or “ameliorating” means a reduction of an undesirable condition or its symptoms. Thus, alleviating or ameliorating includes some reduction, significant reduction, near total reduction, and total reduction.

As used herein, the term “therapeutically effective amount” refers to an amount sufficient to achieve a desired therapeutic effect. The therapeutically effective amount usually refers to the amount administered per injection site per patient treatment session, unless indicated otherwise.

Following long-standing patent law convention, the terms “a,” “an,” and “the” refer to “one or more” when used in this application, including the claims. Thus, for example, reference to “a subject” includes a plurality of subjects, unless the context clearly is to the contrary (e.g., a plurality of subjects), and so forth.

Throughout this specification and the claims, the terms “comprise,” “comprises,” and “comprising” are used in a non-exclusive sense, except where the context requires otherwise. Likewise, the term “include” and its grammatical variants are intended to be non-limiting, such that recitation of items in a list is not to the exclusion of other like items that can be substituted or added to the listed items.

For the purposes of this specification and appended claims, unless otherwise indicated, all numbers expressing amounts, sizes, dimensions, proportions, shapes, formulations, parameters, percentages, quantities, characteristics, and other numerical values used in the specification and claims, are to be understood as being modified in all instances by the term “about” even though the term “about” may not expressly appear with the value, amount or range. Accordingly, unless indicated to the contrary, the numerical parameters set forth in the following specification and attached claims are not and need not be exact, but may be approximate and/or larger or smaller as desired, reflecting tolerances, conversion factors, rounding off, measurement error and the like, and other factors known to those of skill in the art depending on the desired properties sought to be obtained by the presently disclosed subject matter. For example, the term “about,” when referring to a value can be meant to encompass variations of, in some embodiments±100%, in some embodiments±50%, in some embodiments±20%, in some embodiments±10%, in some embodiments±5%, in some embodiments±1%, in some embodiments±0.5%, and in some embodiments±0.1% from the specified amount, as such variations are appropriate to perform the disclosed methods or employ the disclosed compositions.

Further, the term “about” when used in connection with one or more numbers or numerical ranges, should be understood to refer to all such numbers, including all numbers in a range and modifies that range by extending the boundaries above and below the numerical values set forth. The recitation of numerical ranges by endpoints includes all numbers, e.g., whole integers, including fractions thereof, subsumed within that range (for example, the recitation of 1 to 5 includes 1, 2, 3, 4, and 5, as well as fractions thereof, e.g., 1.5, 2.25, 3.75, 4.1, and the like) and any range within that range.

### EXAMPLES

The following Examples have been included to provide guidance to one of ordinary skill in the art for practicing representative embodiments of the presently disclosed subject matter. In light of the present disclosure and the general level of skill in the art, those of skill can appreciate that the following Examples are intended to be exemplary only and that numerous changes, modifications, and alterations can be employed without departing from the scope of the presently disclosed subject matter. The synthetic descriptions and specific examples that follow are only intended for the purposes of illustration, and are not to be construed as limiting in any manner to make compounds of the disclosure by other methods.

### Example 1

### Sustained IGF-1 Delivery Ameliorates Effects of Chronic Denervation and Improves Functional Recovery After Peripheral Nerve Injury

### 1.1 Overview

Functional recovery following peripheral nerve injury is limited by progressive atrophy of denervated muscle and Schwann cells (SCs) that occurs during the long regenerative period prior to end-organ reinnervation. Insulin-like growth factor 1 (IGF-1) is a potent mitogen with well-described trophic and anti-apoptotic effects on neurons, myocytes, and SCs. Achieving sustained, targeted delivery of small protein therapeutics, such as IGF-1, however, remains a challenge.

To address this need, the presently disclosed matter provides a nanoparticle (NP) delivery system can provide controlled release of bioactive IGF-1 targeted to denervated muscle and nerve tissue to achieve improved motor recovery through amelioration of denervation-induced muscle atrophy and Schwann cell (SC) senescence and enhanced axonal growth or regeneration. Biodegradable NPs with encapsulated IGF-1/dextran sulfate polyelectrolyte complexes were formulated using a flash nanoprecipitation method to preserve IGF-1 bioactivity and maximize encapsulation efficiencies. Under optimized conditions, uniform PEG10k-b-PCL40k NPs were generated with an encapsulation efficiency of 88.4%, loading level of 14.2%, and a near-zero-order release of bioactive IGF-1 for more than 20 days in vitro.

The effects of locally delivered IGF-1 NPs on denervated muscle and SCs were assessed in a rat median nerve transection-without-repair model. The effects of IGF-1 NPs on axonal growth or regeneration, muscle atrophy, reinnervation, and recovery of motor function were assessed in a model in which chronic denervation is induced prior to nerve repair. IGF-1 NP treatment resulted in significantly greater recovery of forepaw grip strength, decreased denervation-induced muscle atrophy, decreased SC senescence, and improved neuromuscular reinnervation.

A nanoparticle delivery system was engineered that provides sustained release of bioactive IGF-1 for at least 20 days in vitro; and demonstrated in vivo efficacy in a translational animal model. IGF-1 targeted to denervated nerve and muscle tissue provides significant improvement in functional recovery by enhancing nerve regeneration and muscle reinnervation while limiting denervation-induced muscle atrophy and SC senescence. Targeting the multimodal effects of IGF-1 with a novel delivery strategy may represent the first clinically translatable therapy to improve functional outcomes in patients with peripheral nerve injuries. The NP formulation process is scalable, and the components used for encapsulation are commonly used for other clinical applications. The presently disclosed delivery system for therapeutic small proteins has broad potential applicability beyond this indication.

### 1.2 Background

Although peripheral nerve injuries (PNIs) are common, with an estimated incidence of 68,000 major PNIs occurring each year in the United States alone, Taylor et al., 2008, there are currently no therapeutics available to improve functional recovery in patients with PNI. A number of experimental agents, Chan et al., 2014, have demonstrated the ability to accelerate axonal growth or regeneration and thereby shorten the duration of denervation in animal studies. An optimal therapeutic approach to maximize functional recovery, however, also would directly support denervated muscle and SCs prior to reinnervation.

Insulin-like growth factor 1 (IGF-1), the primary effector of the growth hormone (GH) axis, is a potent mitogen with well-described trophic and anti-apoptotic effects on neurons, Feldman et al., 1997; Kanje et al., 1989; Apel et al., 2010; Ishii et al., 1994, myocytes, Shavlakadze et al., 2005: Stitt et al., 2004; Bodine et al., 2001; Heszele and Price, 2004, and SCs, Delaney et al., 2001; Schumacher et al., 1993; Yan et al., 2018; Cheng et al., 2000. IGF-1 has the potential to improve functional outcomes in a multimodal fashion by augmenting axonal growth or regeneration while also independently acting on muscle and SCs to ameliorate the deleterious effects of chronic denervation. Tuffaha et al., 2016a; Ishii et al., 1993; Tuffaha et al., 2016b; Bianchi et al., 2017; Saceda et al., 2011; Lopez et al., 2019.

Upregulation of endogenous IGF-1 with systemic growth hormone therapy has shown promise in translational models for PNI. Tuffaha et al., 2016a; Tuffaha et al., 2016b; Lopez et al., 2019. This strategy, however, is limited by the requirement for daily parenteral dosing and unwanted systemic toxicities. Furthermore, systemic GH therapy may not provide maximal efficacy given limitations in the extent to which local endogenous IGF-1 tissue concentrations can be upregulated by modulating GH signaling.

In contrast, local delivery of IGF-I can avoid systemic distribution and daily parenteral injections while allowing for controlled dose titration to achieve maximal efficacy. Local infusion of IGF-1 at the site of nerve injury enhances axonal growth or regeneration, muscle reinnervation, and return of motor function in animal models for nerve injury-and-repair. Apel et al., 2010: Sjoberg and Kanje, 1989; Kanje et al., 1991; Emel et al., 2011: Tiangco et al., 2001: Fansa et al., 2002. These studies, however, relied on implanted infusion pumps to achieve sustained, local delivery of IGF-1, which is impractical and unlikely to be adopted for human clinical use.

A safe, effective, and practical delivery strategy for sustained local delivery of IGF-1 targeted to nerve and muscle tissue is therefore needed. Because IGF-1 is a small protein therapeutic, it poses special challenges in achieving this aim as small proteins are particularly susceptible to conformational change and denaturation in non-physiological environments. While progress has been made in engineering delivery systems for small molecule therapeutics, none of the previously described approaches adequately addresses the performance criteria for small protein therapeutics delivery: payload capacity, bioactivity retention, and release control.

Hydrogels that provide a hydrophilic environment for proteins can maintain protein bioactivity, but the release rate tends to be very fast unless chemical conjugation is added between the protein and hydrogel, Tae et al., 2006: Wang et al., 2018, or the protein is directly used as the building block for hydrogel formation. Tang et al., 2018. These modifications, however, alter the composition of the protein into a different chemical species necessitating additional FDA approval for translational purposes. Nanoscale hydrogels formed via polyelectrolyte complexation, He et al., 2019; Xu et al., 2017, suffer from rapid payload release and tend to provide less stability and bioactivity retention of the therapeutic protein.

Biodegradable nanocarrier delivery systems can achieve prolonged local release of therapeutic agents to target tissues. Majumder et al., 2019. Development of a delivery system that can provide sustained release of small proteins for an extended duration, however, has yet to be achieved. Previously reported methods for encapsulating growth factors either release the payload rapidly or achieve prolonged presence through covalent conjugation, which limits capacity and increases susceptibility to protein loss via surface erosion. Li et al., 2001; Park et al., 2012. The relatively low molecular weight and high-water solubility of IGF-1 presents an even greater challenge to be encapsulated in the polymer-based delivery systems.

Flash nanoprecipitation (FNP) is a versatile technique to generate polymeric core-shell NPs; however, water-soluble bioactive proteins, like IGF-1, exhibit poor partitioning into hydrophobic nanocarrier cores and are susceptible to denaturation. Johnson, R. K. Prud'homme, 2003. A solvent reversal process, termed inverse flash nanoprecipitation (iFNP), was developed to facilitate the encapsulation of hydrophilic drugs. Markwalter and Prud′homme, 2018. This process, however, does not sufficiently preserve bioactivity, exhibits poor loading capacity, and the hydrophobic outer shell of inverted NPs must undergo further processing to be suitable for in vivo administration. Markwalter et al., 2020.

To overcome these challenges, the presently disclosed subject matter employs polyelectrolyte complexation (PEC) to stabilize IGF-1 in its bioactive state and render it more compatible for heterogenous nucleation to facilitate FNP encapsulation. The design and optimization of this encapsulation method to generate injectable, biodegradable IGF-1 NPs using PEC and FNP is reported herein. The manufacturing process was optimized by characterizing the effects of solvent, flow rate, O/W ratio, NP composition and other process parameters on payload capacity and protein release kinetics to optimize IGF-1 delivery. The delivery system was tested in a newly validated translational rodent model that enables accurate measurement of functional recovery over time and models the deleterious effects of chronic denervation that impede clinical outcomes following PNI. The presently disclosed subject matter tests, in part, the hypotheses that (1) this NP delivery system can provide controlled, sustained release of bioactive IGF-1 to target nerve and muscle; and (2) IGF-1 NP treatment enhances axonal growth or regeneration and muscle reinnervation while mitigating the deleterious effects of chronic denervation to thereby improve functional recovery in patients following PNI.

### 1.3 Results and Discussion

1.3.1. Design. Fabrication, and In Vitro Characterization of IGF-1 Nanoparticle Delivery System
1.3.1.a. Process Design and Stepwise Optimization of IGF-1 Nanoparticle Formulation

The translation of NP-mediated delivery of water-soluble bioactive protein therapeutics has, to date, been limited in part by the complexity of the fabrication strategies. FNP is commonly used to encapsulate hydrophobic therapeutics, offering a simple, efficient, and scalable technique that enables precise tuning of particle characteristics. Xu et al., 2017. Although the new iFNP process improves water-soluble protein loading, it is difficult to preserve the bioactivity of encapsulated proteins with this approach. Markwalter and Prud'homme, 2018.

To address these limitations, a pre-formulation step was introduced to assemble PECs by complexed coacervation of positively charged IGF-1 and dextran sulfate (DS) polyanions. PECs were then suspended in dimethyl sulfoxide (DMSO), which was selected based on its ability to resuspend PECs. Among several solvents tested, including acetonitrile (ACN), tetrahydrofuran (THF), and dimethylformamide (DMF), IGF-1/DS PEC complexes were fully soluble in DMSO, whereas the other solvents only partially dissolved the PEC.

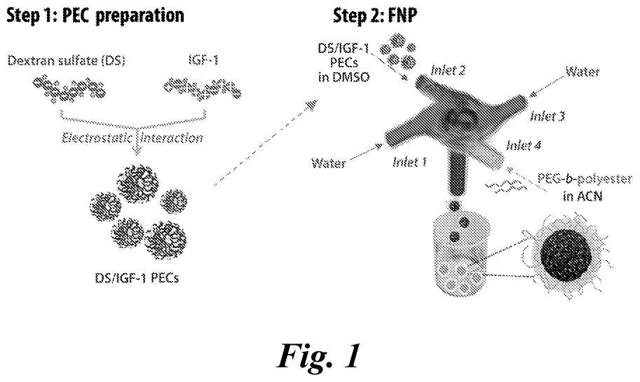

The DMSO/PEC solution was then introduced into a 4-inlet vortex micromixer simultaneously with a PEG-b-PLGA/ACN solution and two water jets (FIG. 1). The solvent polarity change, as a result of rapid mixing of DMSO, ACN and water, induced precipitation of the PLGA segments and assembly of PEG-b-PLGA NPs. Through this process, DS/IGF-1 PECs were encapsulated into PEG-b-PLGA NPs as they phase-separated together. The pre-formulation of IGF-1 into the PEC form proved critical in achieving a high degree of encapsulation (greater than 80%). In contrast, using noncomplexed, native IGF-1 resulted in poor encapsulation (3.6%).

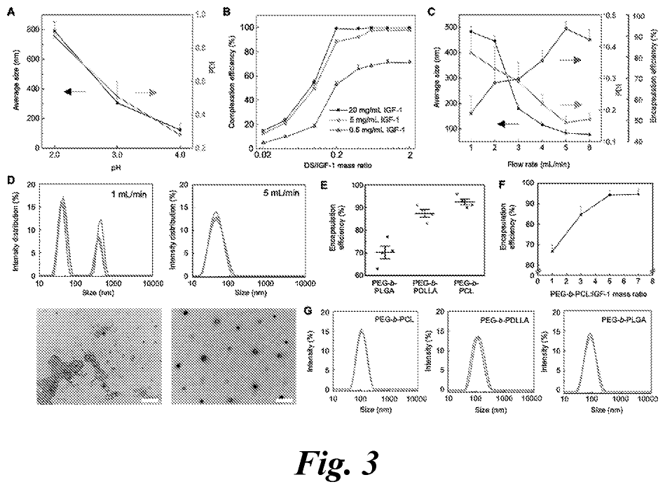

Experimental parameters were optimized sequentially to maximize complexation of IGF-1 with DS. The pH of the IGF-1 solution was lowered, thereby increasing the net positive charge of IGF-1 at the point of complexation. Complete complexation (greater than 99%) was observed at pH<4; however, larger and more heterogenous particles were observed when the pH was lower, which compromised subsequent encapsulation into PEG-b-PLGA NPs (FIG. 3A). Therefore, a pH of 4.0 was selected for the DS/IGF-1 PEC formation step.

To further optimize complexation efficiency, the IGF-1 concentration and counterion ratio were experimentally varied. Complexation efficiencies improved as the IGF-1 concentration increased (FIG. 3B). When adjusting the counterion ratio, complexation efficiencies improved as the DS:IGF-1 ratio increased until a plateau was reached at stoichiometric equivalence. Subsequent increases in DS concentration did not improve the complexation efficiency, nor did it influence the composition of the PECs. Thus, IGF-1/DS PECs were prepared at pH 4 with an IGF-1 concentration of 20 mg/ml and a DS/IGF-1 mass ratio of 0.2 for subsequent experiments. The prepared PECs were collected by centrifugation and lyophilized for subsequent encapsulation.

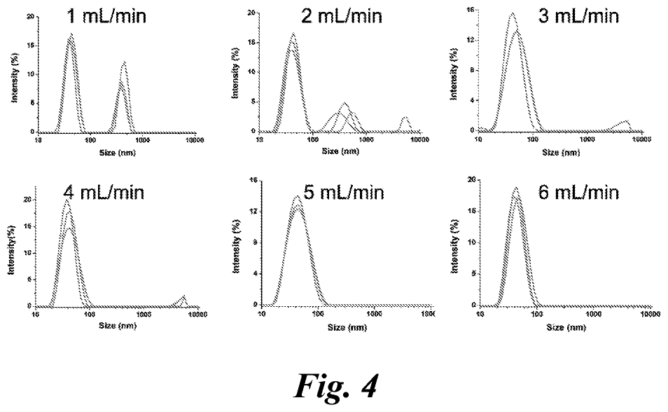

IGF-1/DS PECs were dispersed in DMSO and introduced into a 4-inlet vortex mixer for FNP (FIG. 1, Step 2). The flow rate significantly affected the encapsulation efficiency, which peaked at 88.4% with a flow rate of 5 mL/min (FIG. 3C). Similarly, increasing flow rates generated more uniform and smaller NPs (FIG. 3D, FIG. 4). These observations are consistent with the literature reports which postulate that higher flow rates improve mixing efficiency within the vortex chamber. He et al., 2018; He et al., 2019.

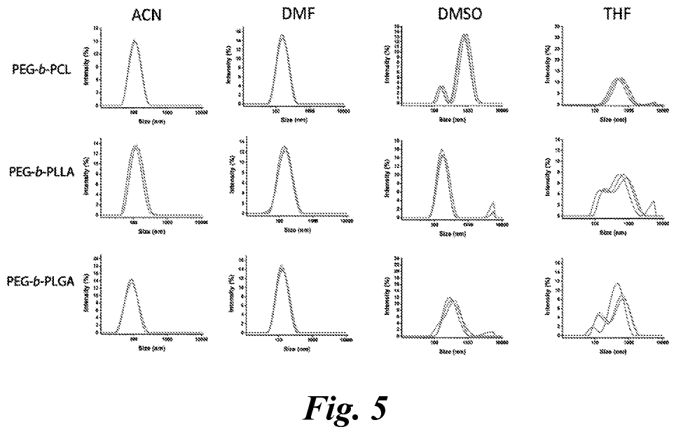

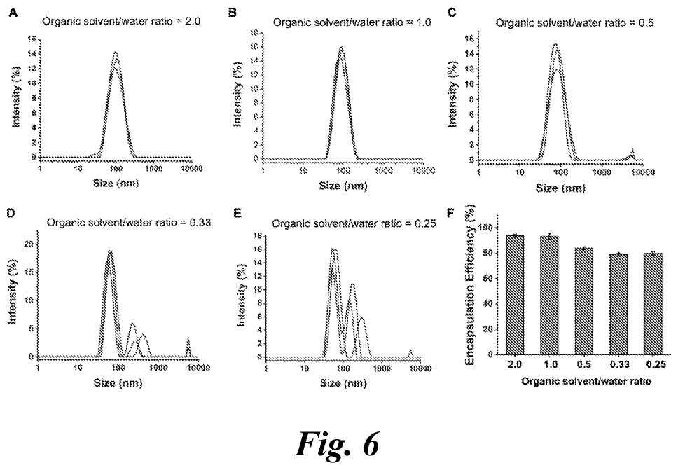

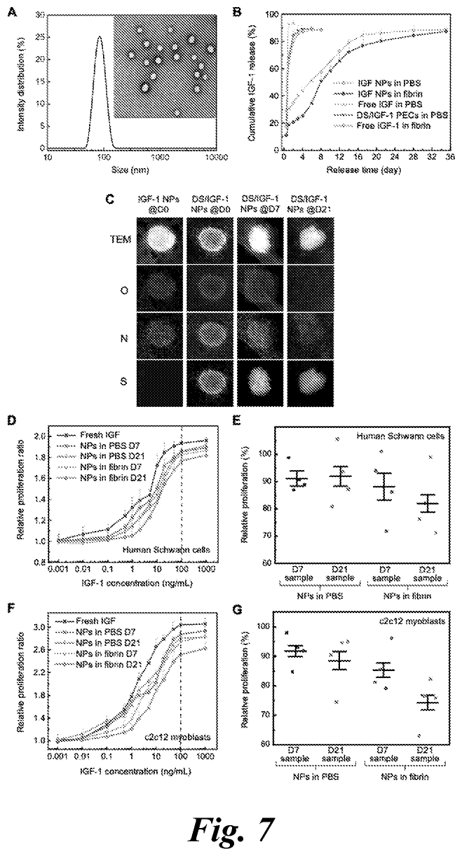

Next, various biodegradable polymers and water-miscible solvents were evaluated to encapsulate DS/IGF-1 PECs (FIG. 5). Three amphiphilic block copolymers, PEG-b-PDLLA, PEG-b-PCL, and PEG-b-PLGA, all exhibited sufficient NP formation and encapsulation efficiencies (FIG. 3E-G). Using ACN or DMF as the solvent for these copolymers generated the most uniform NPs (FIG. 6). DMSO and THE solvents exhibited more heterogenous phase separation and produced large aggregates. Using ACN as a solvent, PEG-b-PCL yielded the highest encapsulation efficiency with a polymer to IGF-1 ratio ≥5.

Based on these findings, the optimized encapsulation parameters for the second step FNP process were: 2 mg/mL of PECs in DMSO, 10 mg/mL of PEG-b-PCL in ACN (5:1 ratio of PEG-b-PCL to IGF-1), and a flow rate of 5 mg/mL for all four jets. The assembled NPs displayed an intensity-average hydrodynamic size of 53.4±1.4 nm with a PDI of 0.13±0.02, and an average zeta potential of −20.8±2.6 mV (FIG. 7A). The average encapsulation efficiency of IGF-1 in the PEG-b-PCL NPs was 88.4±3.9%. The average loading level of IGF-1 in the PEG-b-PCL NPs was 14.2±0.9%.

1.3.1.b. In Vitro Release Characterization of IGF-1-loaded Biodegradable Nanoparticles

The in vitro release profiles were evaluated via dialysis sampling. Non-encapsulated DS/IGF-1 PECs exhibited 70% burst release on day 1 and plateaued at day 3 (FIG. 7B). This observation suggests the release of IGF-1 from its PEC form occurs through dissociation triggered by charge screening in the release medium. DS/IGF-1 PEC-loaded NPs yielded a sustained release of IGF-1 for over 20 days. Without wishing to be bound to any one particular theory, it is thought that this gradual release was due to the slow diffusion of water through the hydrophobic PCL matrix in the core of NPs, producing a more sustained release profile (FIG. 7C).

Of note, in the in vivo study, NPs were mixed into a fibrin gel as a carrier to maintain the NPs around the nerve stump and minimize the loss of NPs from the target tissue. Therefore, the release characteristics of the NPs in fibrin gel also were assessed. IGF-1-loaded NPs embedded in fibrin exhibited a slight delay in the release of IGF-1. This slight delay also was observed when free, non-encapsulated IGF-1 was embedded in fibrin gel. This observation is likely due to the protein-protein interaction between IGF-1 and fibrin, as reported in the literature. Hunter and Hers, 2009.

The bioactivity of released IGF-1 was evaluated by measuring its mitogenic activity in Schwann cell (SC) and myoblast cultures. Compared to fresh IGF-1, protein released from NPs at day seven and day 21 retained 91.8±3.1 and 92.6=1.9% bioactivity, respectively (FIG. 7D, FIG. 7E, FIG. 7F, FIG. 7G). This bioactivity was modestly reduced when NPs were embedded in a fibrin carrier. The preserved bioactivity is likely the result of the unreleased IGF-1 within the NPs being protected in a condensed, complexed form, which shields it from denaturation attack (in vitro) and enzymatic cleavage (in vivo).

In FIG. 7D and FIG. 7F, the Schwann cell (SC) and myocyte proliferation assays demonstrated a maximum proliferation pattern at an IGF-1 concentration greater than 100 ng/ml. The total volume of the forelimb is about 2 mL to 3 mL, based on weight measurement (the density of muscle tissue is estimated to be about 1 g/mL). A minimum delivery rate of 300 ng IGF-1 per day was calculated to achieve maximal efficacy in the rat PNI model. Given that therapeutic proteins have 20% or less bioavailability, it was estimated that 30 μg of encapsulated IGF-1 released over 20 days would achieve an effective daily dose of about 1.5 μg/day in each rat, which is in line with prior studies. Yan et al., 2018.

### 1.3.2. Evaluation of In Vivo Release and Efficacy

1.3.2.a. Translational Rodent Model for PNI with Induced Chronic Muscle and Nerve

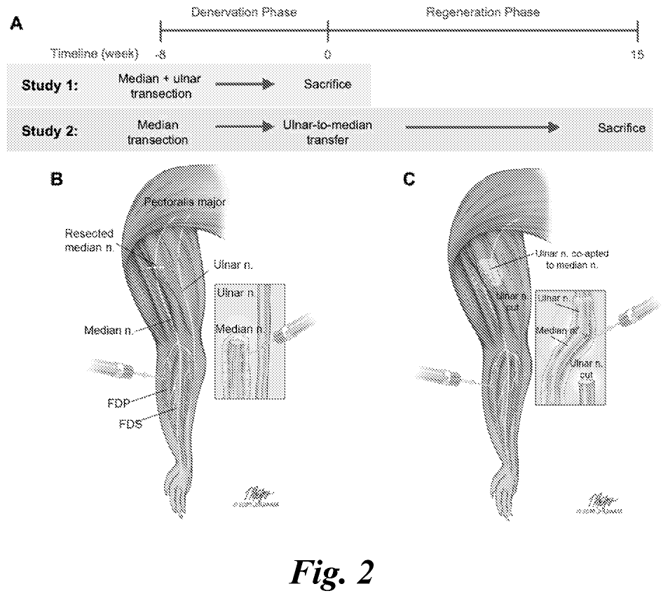

Denervation and Reliable Measurement of Functional Recovery over Time. The treatment effects of optimized IGF-1 NPs on nerve regeneration, muscle reinnervation and functional recovery were evaluated in a rat PNI model in which chronic denervation of the distal nerve track and muscle is induced prior to nerve repair. Lopez et al., 2019. The median nerve was transected and left unrepaired for eight weeks, followed by delayed nerve repair. The second stage repair involved proximal ulnar-to-distal median nerve transfer (FIG. 2) such that the axons from the ulnar nerve regenerated through the chronically-denervated distal median nerve pathway and into chronically denervated muscle (Table 1).

TABLE 1
Experimental design for investigating the effect of local
IGF-1 delivery on denervation and repair in two rat models

| | | | |
|---|---|---|---|
| Group | Surgical intervention (s) | Treatment | Endpoint |

Study 1: Median and ulnar nerve transection without repair

| Positive | Median + ulnar nerve transection | Empty NP | Week 1 |
|---|---|---|---|
| control | | vehicle | (n = 8) |
| Negative | | Empty NP | Week 8 |
| control | | vehicle | (n = 8) |
| Experimental | | IGF-1 NPs | Week 8 |
| group | | | (n = 8) |

Study 2: Delayed ulnar-to-median nerve transfer with repair

| | | | | |
|---|---|---|---|---|
| Positive | Sham | Ulnar-to-median | Empty NP | Week 15 |
| control | | nerve transfer | vehicle | (n = 8) |
| Negative | Median | | Empty NP | Week 15 |
| control | nerve | | vehicle | (n = 8) |
| | transection | | | |
| Experimental | Median | | IGF-1 NPs | Week 15 |
| group | nerve | | | (n = 8) |
| | transection | | | |

In rodent models, delaying nerve repair for a period of time to allow for chronic denervation to occur is necessary to produce the degree of denervation-induced muscle and SC atrophy that occurs following immediate repair of PNI in humans; this is because the regenerative distances are substantially greater in humans than in rodents. Sulaiman and Gordon, 2000. The first-described rodent PNI model with induced chronic denervation involves tibial nerve transection prior to delayed peroneal-to-tibial nerve transfer. The major limitation of this model is that it does not provide reliable measurement of functional recovery over time, which is the critical measure of efficacy when evaluating therapeutics to enhance peripheral nerve regeneration.

Isometric tetanic force testing (ITFT), often used with this model, is a terminal procedure that can be performed only once at the time of sacrifice. In contrast, the presently disclosed median nerve injury model with induced chronic denervation allows for use of a functional assay that was recently validated that provides comparable reliability to ITFT while also providing serial in vivo measurements. Hanwright et al., 2019.

IGF-1 NPs suspended within fibrin or vehicle alone were administered extraneurally at the nerve repair site and along the distal median nerve, as well as intramuscularly within the denervated muscle, to target treatment to the regenerating axons and denervated SCs and muscle, respectively. Initial dosing was performed at the time of median nerve transection, with interval re-dosing every 2 weeks thereafter.

1.3.2.b. IGF-1 NPs Reduced Denervation-induced Muscle and Schwann Cell Atrophy

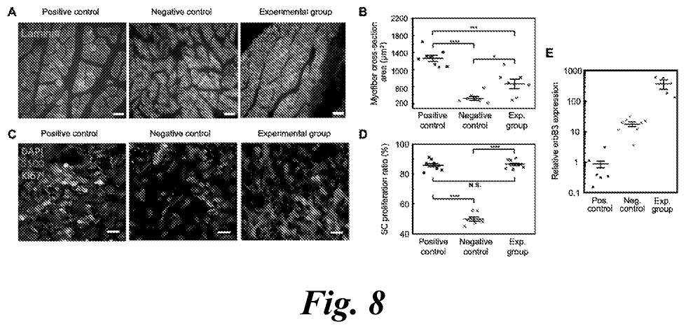

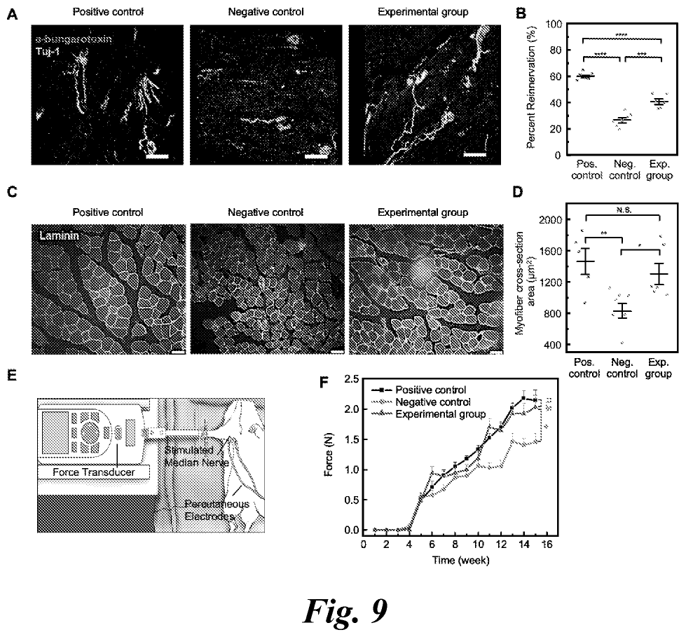

To isolate the treatment effects on muscle and SC atrophy, independent of axonal growth and reinnervation, cohorts of IGF-1-treated experimental animals and untreated negative control animals were sacrificed following the 8-week denervation period without undergoing subsequent nerve repair (FIG. 2, Study 1). Muscle denervation causes progressive loss of myofibril size, limiting the extent of motor functional recovery when reinnervation occurs. Carlson et al., 1996. Myofiber cross-sectional area (CSA) of the median nerve-innervated forearm flexor muscles was used to quantify the degree of denervation-induced muscle atrophy. IGF-1 NP treatment was found to significantly decrease muscle atrophy during the period of denervation (FIG. 8A, FIG. 8B).

1.3.2.c. IGF-1 NPs Maintained SC Proliferation During Denervation

SCs provide critical support to regenerating axons. During Wallerian degeneration, SCs proliferate to secrete neurotrophic factors and form longitudinally oriented Bands of Bünger that provide structural support to regenerating axons. With chronic denervation, however, SCs senesce and lose the capacity to proliferate and support regenerating axons. Ishii et al., 1993; Glanzer and Ishii, 1995.

To evaluate the trophic effects of IGF-1 NP treatment on denervated SCs, a portion of the median nerve distal to the injury site was harvested following an eight-week period of denervation (FIG. 2, Study 1). Proliferating SCs were identified by positive co-staining of S100 and Ki67. There was increased Ki67 co-staining in the IGF-1 treated cohort compared to negative controls (86.5±2.8% vs. 50.0±3.7%, respectively, p<0.05, FIG. 8C, FIG. 8D). Similarly, ErbB3 expression was up-regulated 19-fold in the experimental cohort relative to negative controls (p<0.05, FIG. 8E).

ErbB3 is a SC receptor for neuregulin that induces proliferation and is downregulated during chronic denervation. Park et al., 2012. These findings support the hypothesis that IGF-1 NP treatment maintains denervated SCs in a proliferating state so that they can better support regeneration. These findings are consistent with prior reports that IGF-1, via the PI3-K/AKT pathway, increases SC expression of myelin basic protein, myelin associated glycoprotein and DNA synthesis to promote survival, proliferation and differentiation to a myelinating phenotype once regenerated. Delaney et al., 2001; Stewart et al., 1996; Fex Svenningsen and Kanje, 1996.

### 1.3.2.3. IGF-1 NPs Enhanced Motor Endplate Reinnervation

Terminal axons must ultimately reinnervate motor end plates within target muscles to reconstitute functional neuromuscular junctions (NMJs) (FIG. 9A, FIG. 9B). To enumerate the rate of reinnervation, intact NMJs were quantified via the co-detection of a presynaptic neuronal marker (TUJ1) and the postsynaptic acetylcholine receptor marker alpha-bungarotoxin (α-BTX) following the 15-week regeneration period (FIG. 2, Study 2). In addition, IGF-1 NP treated animals exhibited larger myofiber CSAs as compared to untreated negative controls (1307±135 vs. 834±95 μm², respectively, p<0.001, FIG. 9C). The IGF-1 treated animals exhibited increased rates of NMJ reinnervation compared to negative control animals (40.9±5% vs. 26.9±5%, respectively, p<0.05, FIG. 9D). This observation is likely due to the combined trophic effects of IGF-1 on proliferating SC and regenerating axons, which together would manifest in a greater number of axons reaching the target muscle. IGF-1 also is known to promote sprouting into muscle: therefore, beyond improved axonal growth or regeneration, motor unit expansion might also have contributed to the differences observed in NMJ reinnervation. Caroni and Grandes, 1990.

1.3.2.3.e. IGF-1 NPs Improved Functional Recovery

Motor functional recovery is the primary outcome of interest, as it reflects the cumulative upstream treatment effects and captures the ultimate clinical goal of treatment. Motor function was measured weekly during the 15-week regeneration period (FIG. 2, Study 2) with stimulated grip strength testing (SGST) (FIG. 9E) that was developed and validated previously. Hanwright et al., 2019. Motor recovery was first detectable at a median of 5 weeks in all groups (p>0.05), suggesting that IGF-1 treatment did not appreciably hasten initial reinnervation, perhaps because the regenerative distance from the repair site to the most proximal muscle branches was quite short in the rodent model (FIG. 9F). More forceful grip strength, however, was recovered in IGF-1 treated animals at weeks 11, 12, 14, and 15. Importantly, motor functional recovery in the IGF-1 treated cohort was comparable to that of the positive control animals that were not subjected to a period of denervation. This observation supports the notion that IGF-1 NPs can ameliorate the damaging effects of denervation-induced muscle atrophy and SC senescence and is consistent with recent findings from Raimondo et al., 2019, which showed improved functional recovery in rabbits with locally administered VEGF and IGF-1 via a calcium alginate hydrogel.

Consistent with prior translational studies assessing local IGF-1 delivery via mini-pump, Apel et al., 2010: Sjoberg and Kanje, 1989; Kanje et al., 1991: Emel et al., 2011: Tiangco et al., 2001: Fansa et al., 2002, the present findings of greater reinnervation of motor endplates and improved functional recovery suggest improved axonal growth or regeneration with IGF-1 NP treatment. Retrograde labelling, however, would be helpful in future studies to delineate the number of motor neurons contributing to end-organ reinnervation, as IGF-1 is known to promote sprouting into muscle, Caroni and Grandes, 1990, and motor unit expansion may contribute to these observations.

1.3.2.f. Measured Local and Systemic Levels of IGF-1 Delivered by NPs

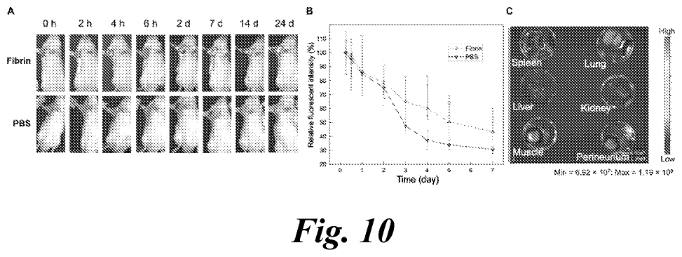

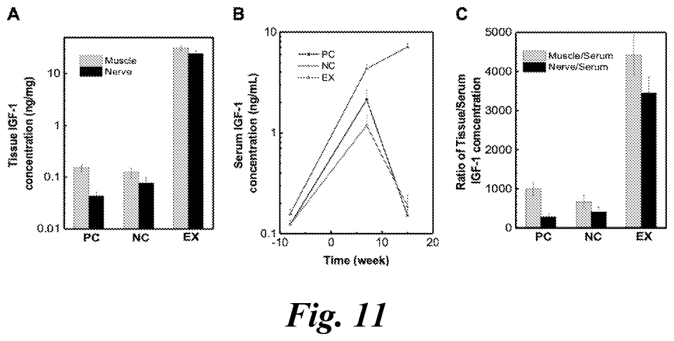

Local and systemic IGF-1 concentrations were evaluated. Without wishing to be bound to any one particular theory, it was thought that local IGF-1 therapy would reduce systemic distribution and associated toxicities. Despite the targeted local administration, however, there remained the possibility that IGF-1 delivered to muscle would be taken up into the blood stream. Encouragingly, minimal systemic distribution was observed relative to the elevations observed in local tissue concentrations, with local tissue concentrations approximately 3,000 times greater than serum concentrations in IGF-1 treated animals at the study endpoint (FIG. 10, FIG. 11). The extent of elevation in local IGF-1 concentration is much greater than what was observed in other studies demonstrating efficacy with GH and local IGF-1 treatment, Lopez et al., 2019, raising the possibility that lower doses might have achieved maximal efficacy with even less systemic distribution. Future dose-response studies are needed to evaluate this question further.

### 1.4 Summary

To realize the therapeutic potential of IGF-1 treatment for PNIs, a novel local delivery system for small proteins using a new FNP-based encapsulation method that offers favorable encapsulation efficiency with retained bioactivity and a sustained release profile for over 3 weeks was designed, optimized, and characterized. The IGF-1 NPs demonstrated favorable in vivo release kinetics with high local loading levels of IGF-1 within target muscle and nerve tissue. Consistent with prior studies, IGF-1 treatment minimized denervation-induced muscle atrophy and SC senescence and improved axonal growth or regeneration and NMJ reinnervation, leading to greater recovery of motor function.

The new small animal PNI model used in this study, Hanwright et al., 2019, replicates the deleterious effects of chronic muscle and SC denervation that are the primary cause of poor clinical outcomes in humans and provides accurate and reliable measurement of motor functional recovery as the primary outcome of interest. As such, it will serve as an ideal translational model to evaluate future interventions aimed at improving nerve regeneration and functional recovery following PNI.

The NP formulation method developed in this study provides steady release of bioactive IGF-1 over 3 weeks. Further, the injectability of the formulation permits redosing at the local site relatively easily. The FNP method developed here for IGF-1 encapsulation has the advantages of high efficiency of encapsulation, high uniformity of the NPs, sustained release profile, and high bioactivity retention. These unique features are enabled by the new FNP-based encapsulation method, which achieves IGF-1 loading into PEG-b-polyester NPs by using a pre-formulated IGF-1/DS PECs, which also are insoluble in the NP assembly solution (DMSO/ACN/water mixture, 1:1:2, v/v), therefore being encapsulated in the hydrophobic core of the NPs. The flash precipitation process permits supersaturation of the PECs in NP core, as a result of a kinetically arrested entrapment mechanism. The use of IGF-1/DS PECs, instead of free IGF-1, provides protection of IGF-1 against denaturation during NP preparation under high shear flow condition, and resists degradation in vivo throughout the release/treatment duration, as IGF-1 is retained in a condensed state analogous to the transient complexed conformation of many functional proteins during their transport processes.

Achieving a 3-week duration of bioactive IGF-1 represents an important advance in small protein delivery: for comparison, a recent study evaluating local IGF-1 therapy for PNI using an alginate delivery system achieved only approximately 5 days of sustained IGF-1 release. Raimondo et al., 2019. The platform described herein, however, has the potential for further improvement to extend the release duration. In the present study, fibrin gel was used as a readily available carrier for in vivo delivery. A more elaborate delivery carrier may be developed to better protect the NPs for the duration of release and potentially enhance the release kinetics inherent to the NPs.

Both the therapeutic agent itself and the novel method by which it is fabricated are promising due to their significant potential for clinical translation and scalability. IGF-1 is FDA-approved for the treatment of primary IGF-1 deficiency and the polymer components of NP formulation, DS and PEG-b-PCL, are currently used in FDA-approved formulations and devices, which will facilitate clearance of regulatory hurdles. The NP formulation method is amenable to large scale production with high uniformity and reproducibility. With regard to clinical application, the IGF-1 NPs can be delivered into denervated muscle and around the injured nerve at the time of surgical repair, with interval re-dosing under ultrasound guidance. Beyond facilitating IGF-1 treatment for PNI, the encapsulation method described in this study has broad potential applicability beyond to a multitude of clinical scenarios in which sustained, targeted delivery of therapeutic small proteins may be beneficial.

### 1.5 Materials and Methods

### 1.5.1 NP Formulation

Human recombinant IGF-1 was reconstituted in deionized (DI) water (20 mg/mL, pH 5.0; BioVision). PECs were formed with DS (100 mg/mL) and were resuspended in DMSO at 2 mg/mL in a 5-mL syringe (FIG. 1). PEG10k and PCL40k co-polymers in ACN (10 mg/mL) were loaded in a separate 5-mL syringe. A 4-inlet vortex mixer was loaded with the polymer and PEC solutions and two DI water streams and feed into the mixing chamber at 10 mL/min to yield PEG-b-PCL micellar NPs through rapid micro-mixing FNP. The NP solution was dialyzed through a dialysis membrane (MW 3.4 kD, SpectrumLab) against DI water for 6 h with the water changed every 30 min. Dynamic light scattering (DLS) and transmission electron microscopy (TEM) was used to assess NP size distribution and morphology. Encapsulation efficiency and loading capacity were quantified by dialyzing 1 mL NP solution against 100-fold DI water for 12 h in a Float-A-Lyzer (MW 1000 kD, SpectrumLab). MicroBCA (ThermoFisher) measured the concentration of unencapsulated IGF-1 that diffused outside the Float-A-Lyzer.

### 1.5.2 In Vitro Release Kinetics and Bioactivity

NPs containing 300 μg of IGF-1 in 1 mL phosphate-buffered saline (PBS, pH 7.4) were loaded in a dialysis tube (SpectrumLab) and were incubated at 37° C. at 100 rpm. To evaluate the fibrin embedded construct, the NP PBS solution was combined with fibrin gel (35 mg fibrinogen, 9 mg NaCl in 1 mL of NP suspension, crosslinked with 10 KIU thrombin) and placed in separate 50-mL centrifuge tubes with 10 mL and 1 ml PBS. At select times, medium was collected and replaced with PBS. The microBCA assay and a fluorescent plate reader measured IGF-1 concentration in medium from the dialysis tube and fibrin gel, respectively.

Released IGF-1 was lyophilized with 500 mM acetic acid and resuspended at 0.0001, 0.001, 0.01, 0.1, 1, 2, 5, 10, 50, 100 and 1000 ng/mL before being transferred to a 96-well plate with 5,000 C2C12 myoblasts or SCs seeded per well. After incubating for 24 h, 20 μL Alamar Blue assay reagent (ThermoFisher) was added and incubated for 4 h before whole plate analysis by an absorbance plate reader at 570 nm (600 nm reference wavelength).

### 1.5.3 Animal Care and Use

Experimental procedures were performed in accordance with the National Institutes of Health guidelines (NIH Publications #8023) and with approval from the institutional Animal Care and Use Committee (Protocol #18M74). Male Lewis rats aged 4-6 weeks, weighing approximately 160-180 grams were used for all in vivo studies. Surgical procedures and grip strength testing were conducted under general anesthesia using 2.5% inhaled isoflurane. All animals received subcutaneous buprenorphine (0.1 mg/kg) analgesia and enrofloxacin (1 mg/kg) prophylaxis postoperatively.

### 1.5.4 Delivery of IGF-1 NPs In Vivo

Positive and negative control groups received empty NP vehicles and experimental animals received 100 μL of NPs with IGF-1 (300 μg/mL) (FIG. 2 and Table 1). NPs were initially administered every four weeks (weeks-8, −4, 0, 4, 8). Beginning in week 10, dosing was increased to every two weeks (10, 12, and 14). This dosing change was made in light of further in vivo testing that found IGF-1 could only be detect in forearm muscles for two weeks after administration (FIG. 10). To target regenerating neurons and denervated SCs, and NPs in 50-μL fibrin gel were delivered at the coaptation site and exposed distal nerve stump by percutaneous injection with a 27-G hypodermic needle and Leur lock Y-connector (Merit Medical Systems) into the bicipital grove (weeks −4, 4, 8, 10, 12, 14) or under direct vision in surgical procedures (weeks −8, 0). To target denervated muscle, NPs in PBS were injected intramuscularly into the volar forearm flexor muscle compartment percutaneously.

### 1.5.5 Chronic Denervation Without Nerve Repair

To isolate the effects of treatment on SCs and denervated muscle, median and ulnar nerve transection without repair was performed in 24 rats randomized to one of three groups (Table 1). Rats were sacrificed eight weeks postoperatively, and denervated muscle and distal nerve stumps were harvested for analysis. Naïve positive control animals were sacrificed at week one.

### 1.5.6 Characterization of SC Proliferation In Vivo

A 1.0-cm segment of ulnar nerve was harvested from the distal denervated nerve stump and fixed in 4% paraformaldehyde (PFA) overnight, incubated in 30% sucrose (w/v) solution for 48 h at 4° C., and embedded in optimum cutting temperature (OCT) medium. 10 μm cross-sections were cut on a cryostat and permeabilized with 0.2% Triton X-100 (Sigma). 5% goat serum (Sigma) and 20% Tween (Sigma) in PBS blocked nonspecific staining. Primary antibodies against S100 (ThermoFisher, MA1-26621, 1:100) for SCs and against Ki67 (ThermoFisher, RM-9106-S0, 1:400) for proliferating SCs were incubated overnight at 4° C. Secondary antibodies to S100 (Cy5 goat anti-mouse, Jackson Immuno, 115-175-146, 1:600) and Ki67 (Fluorescein goat anti-rabbit, Vector Laboratories, Fl-1000, 1:800) were incubated at room temperature (RT) for 1 h. The total number of SCs (S100) and proliferating SCs (S100/Ki67 co-staining) from immunofluorescent images (Zeiss HAL 100 microscope, Carl Zeiss Microscopy) was counted by a blinded reviewer to determine the percentage of proliferating SCs.

### 1.5.7 Real-Time Quantitative PCR Analysis

To evaluate SC gene expression, 1.0-cm denervated median nerve stump was harvested and snap frozen in liquid nitrogen RNA extraction used a TRIzol® (Invitrogen) and PureLink® RNA mini kit (Invitrogen)-based hybrid protocol. cDNA was synthesized using the QuantiTect® Reverse Transcription Kit (Qiagen). qPCR was performed with the QuantiTect® SYBR Green Kit (Qiagen) for the SC proliferation marker ErbB3 using the housekeeping gene S100B. The 2-AACT method determined fold change of ErbB3 in the experimental group with the negative control as the calibrator. Livak et al., 2001. After the Grubbs' test failed to identify the outliers, the data were analyzed using the Mann-Whitney test as described below.

### 1.5.8 Muscle Atrophy Analysis

The flexor digitorum profundus muscle was harvested following sacrifice and was embedded in October 12-μm axial cross-sections were cut on a cryostat and incubated with 5% Donkey serum in PBS at RT for 1 h, then overnight at 4° C. with 1:500 anti-laminin-γ 1 2E8 (Sigma) primary antibody before incubation at RT with 1:1200 anti-mouse Alexa Fluor 488 IgG (Jackson ImmunoResearch) for 1 h. Myofiber CSA was quantified from immunofluorescent images with MyoVision Basic v1.0 software (University of Kentucky). At least 200 randomly sampled myofibers were counted for each specimen. Wen et al., 2018.

1.5.9 Chronic Denervation with Nerve Repair

A rat chronic denervation forelimb model evaluated functional recovery, Lopez et al., 2019; Kern et al., 2017; Sulaiman and Gordon, 2009. Twenty-four rats were randomized to one of three groups (Table 1) after undergoing median nerve transection without repair (Week-8) before ulnar-to-median nerve transfer (Week 0) for regeneration through the chronically denervated median nerve pathway. Weekly functional testing was performed after nerve repair for 15 weeks.

### 1.5.10 Grip Strength Testing

Stimulated grip strength tests were conducted weekly as previously described, Hanwright et al., 2019, (FIG. 9E). Animals began stimulated grip strength testing one week after nerve transfer. The forelimb was secured in place 90° of abduction and two stimulating needle-point electrodes (ADInstruments) were placed 1 mm apart in the axilla proximal to the insertion of pectoralis major onto the humerus. The transferred ulnar nerve was electrically stimulated supra-maximally at 10 volts at 50 Hz and 100 Hz for maximal tetanic contraction of the median-innervated extrinsic digital flexors. After full digital flexion, a force meter (Chatilon DE II, Ametek) in the palm was distracted away from the animal until the grip was overcome. The greatest mean maximal force generated of three trials at each frequency was used to compare among groups.

### 1.5.11 Neuromuscular Junction Co-immunofluorescence Staining

Co-staining of presynaptic neuronal marker (TUJI) and postsynaptic acetylcholine receptor marker (α-BTX) quantified NMJs in the flexor digitorum sublimis muscle. After PFA fixation, longitudinal sections 50-μm thick were permeabilized with 0.2% Triton X-100 and blocked with 10% goat serum (Sigma) and 0.05% Tween-20 (Sigma) in PBS. Monoclonal mouse anti-β-tubulin III antibody (Sigma T8578, 1:1,000) was incubated overnight in a humidified chamber, after which Alexa 488 donkey anti-mouse antibody (Jackson ImmunoResearch 715-545-151, 1:800) and Alexa Fluor 594 α-BTX (Invitrogen B-13423, 1:1000) were incubated at 37° C. for 1 h before mounting with ProLong® Gold Antifade Mountant with DAPI (Life technologies). Images (Zeiss Axio Imager-2, Carl Zeiss Microimaging) were analyzed by a blinded reviewer with corresponding single band pass filters (Semrock, Rochester, NY) and matched exposure settings. Two spectral windows using ZEN 2012 software were captured for Alexa Fluor 488 (488 nm) and Alexa Fluor 594 (590 nm). Images taken 5 μm apart were flattened into a two-dimensional image and merged. NMJs were classified as denervated (α-BTX only) or occupied (α-BTX/TUJI co-staining). Average (±SD) percentage of occupied NMJs was compared between groups.

### 1.5.12 Measurement of IGF-1 Concentration by ELISA

For IGF-1 in serum, 1 mL whole blood was collected from the dorsal tail vein at weeks-8, 7, and 15 and incubated at RT for 20 min before 10 min of 3000 rpm centrifugation at 4° C. Supernatant was flash frozen and stored at −80° C. For IGF-1 in local tissue, the pronator muscle and 1 cm distal median nerve segment harvested and snap frozen at week 15 were dissected and placed in round bottom microfuge tubes on ice. 300 μL complete extraction buffer (100 mM Tris, pH 7.4, 150 mM NaCl, 1 mM egtazic acid, 1 mM ethylenediaminetetraacetic acid, 1% Triton X-100, 0.5% sodium deoxycholate, phosphatase inhibitor cocktail (1 mL, 50× stock), protease inhibitor cocktail (with phenyl methyl sulfonyl fluoride, Abcam, 250 μL, 500× stock)) was added to each 5-mg piece of tissue before homogenization and shaking at 4° C. for 2 h. The suspensions were combined and centrifuged for 20 min at 13000 rpm at 4° C. and placed on ice. The supernatant was aliquoted to a chilled tube for ELISA (Abcam) according to the manufacturer's protocols.

### 1.5.13 Statistical Analysis

All results are expressed as mean±standard deviation (SD) for each group. Non-parametric Mann-Whitney two-tailed U-test was used to determine significant differences between two groups. A p-value<0.05 was considered statistically significant.

### Example 2

A Translational Drug Delivery Biomaterial for a 6-week Sustained Local Release of IGF-1 to Improve Outcomes Following Nerve Repair

### 2.1 Overview

We previously demonstrated that functional recovery following peripheral nerve injury could be enhanced with local delivery of poly(ethylene glycol)-b-poly(ε-caprolactone) PEG-b-PCL IGF-1 nanoparticles to the regenerating nerve and its target muscle. This treatment ameliorated the deleterious effects of chronic denervation on both the distal stump and target muscle, and significantly improved functional outcomes. We used fibrin gel as a carrier for the IGF-1 nanoparticles to facilitate their administration and localization to the target tissue. We, however, had to repeat the injections every 14 days as the fibrin gel (i.e., fibrin glue) degraded in 10-14 days. This dosing frequency is not clinically favorable.

Accordingly, in some embodiments, the presently disclosed subject matter includes a translational drug carrier system that provides a sustained local release of bioactive IGF-1 for six weeks after a single injection. We first engineered a biocompatible, biodegradable, and mechanically tunable nanofiber-hydrogel composite with specific topographical cues to induce a pro-regenerative environment in nerve/muscle tissue. We further functionalized the composite with IGF-1 nanoparticles, and fine-tuned the release kinetics of bioactive IGF-1 in vitro and in vivo. We determined the optimal in-vivo dosing regimen, and then tested the efficacy of our drug delivery system in a pre-clinical nerve regeneration rat model. We observed that a 6-week injection frequency decreased denervation-induced muscle atrophy and Schwann cell senescence, and improved neuromuscular reinnervation and grip strength. We then investigated the mechanisms through which IGF-1 promoted its trophic effects. We finally tested the translational feasibility of this multimodal, long-lasting therapeutic strategy in a primate model of nerve regeneration.

### 2.2 Background

In Example 1, we tested the efficacy of the IGF-1 NPs in a chronic denervation rat animal model, and noted significantly greater recovery of grip strength, decreased muscle atrophy, decreased SC senescence, and improved neuromuscular reinnervation. We, however, had to repeat the nerve and muscle injections every 14 days since we used fibrin glue as a carrier for the IGF-1 NPs: fibrin degraded in 10-14 days. Hence a more pragmatic carrier that enables the prolonged release of IGF-1 NPs (for the entire period of nerve regeneration) is needed to achieve clinical translatability of our treatment strategy.

### 2.3 Materials and Methods

2.3.1 NHC construction

HA powder was fully dissolved in phosphate buffer saline (PBS) at a pH of 8.5 to achieve a 1% HA solution for 24 hours. Glycidyl acrylate was then added into the HA solution at a volume ratio of 3:100. The mixture was placed on a magnetic stirrer at 37° C. for reaction for 16 hours. After grafting with acrylate groups, modified HA (HA-Ac) was precipitated into ethanol at a 10-fold volume ratio to the HA solution. The precipitate was washed thrice with ethanol and acetone, 30 minutes each time, and then dehydrated with compressed air. The modified HA-Ac was re-dissolved into PBS at pH 7.4 as a stock solution at 20 mg/mL concentration and stored at 4° C. before use. Ellman's Assay determined the modification degree.

The PCL nanofibers with an average length of 20-100 μm were produced by electrospinning. Briefly, poly(ε-caprolactone) (PCL) solution at the concentration of 16% w/w was dissolved in a mixture of dichloromethane (DCM) and dimethylformamide (DMF) at a ratio of 9:1 (w/w). A green-fluorescent dye, poly(9, 9-dioctylfluorene-alt-benzothiladiazole) (F8BT), was added to the PCL solution to label the fibers. The PCL solution was electrospun to form nanofibers. Carboxyl groups were introduced to the surface of the PCL fibers using plasma surface activation. They converted to maleimide (MAL) groups through activation with ethyl dimethylaminopropyl carbodiimide and N-hydroxysuccinimide at a molar ratio of 1:4:4, respectively. N-(2-aminoethyl) MAL was added at a molar ratio of carboxyl groups to amine groups of 1:2, and the mixture was gently shaken to facilitate the conversion. The MAL-modified PCL fibers were broken down into fragments using a cryogenic mill (Freezer/Mill 6770, SPEX SamplePrep, Metuchen, NJ). These fragments were sterilized in three cycles of 70% v/v ethanol followed by distilled water. The resulting sterilized MAL-modified fiber fragments were lyophilized and stored at −20° C. before use.

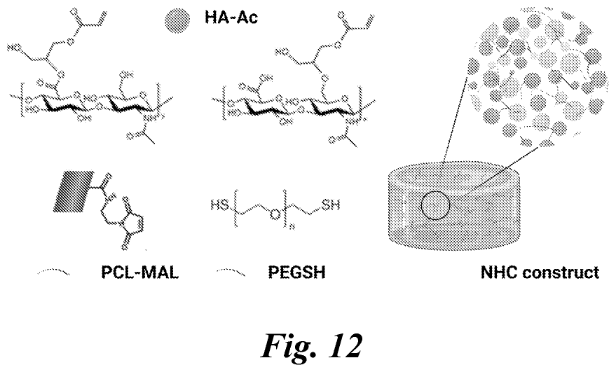

Nanofiber-hydrogel composites were prepared by mixing MAL-fibers in HA-Ac precursor solution with PEGSH. The composites were prepared with 30 mg/ml of MAL-fibers, 10 mg/mL of HA-Ac, and 5 mg/mL PEGSH in phosphate-buffered saline (PBS). The crosslinking method of the composite is a Michael-type addition reaction, which could happen at 37° C. without any additives or by-products (FIG. 12).

### 2.3.2 Characterization of NHC

To investigate the NHC-mediated pro-regenerative response in vivo, NHC and hydrogel controls were injected into the forearm musculature of Lewis rats. Rats were sacrificed 28 days after the injections. Whole animal transcardiac perfusion fixation (through the left ventricle with cold saline followed by 300 ml of 4% paraformaldehyde in 0.1 M PBS, pH 7.4) was used to sacrifice the animals destined for histology and immunohistochemistry (n=4 per group). The animals destined for qRT-PCR were sacrificed with deep anesthesia after extracting the specimen and immediately freezing it (n=6 per group).

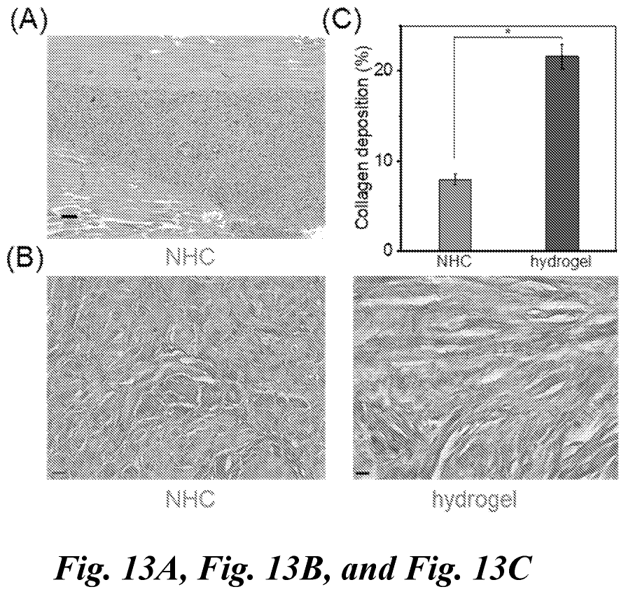

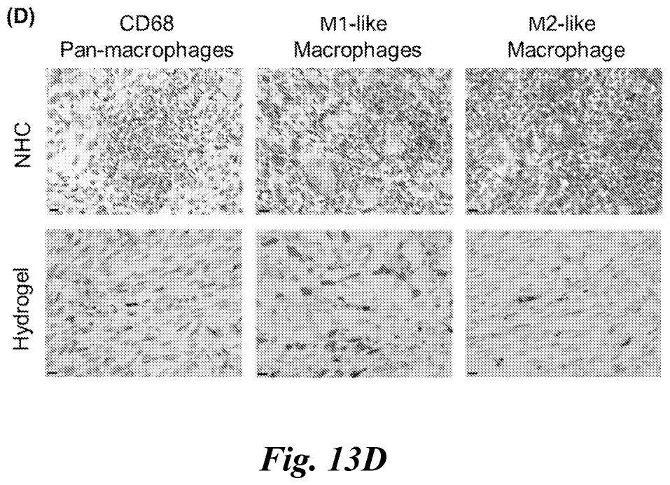

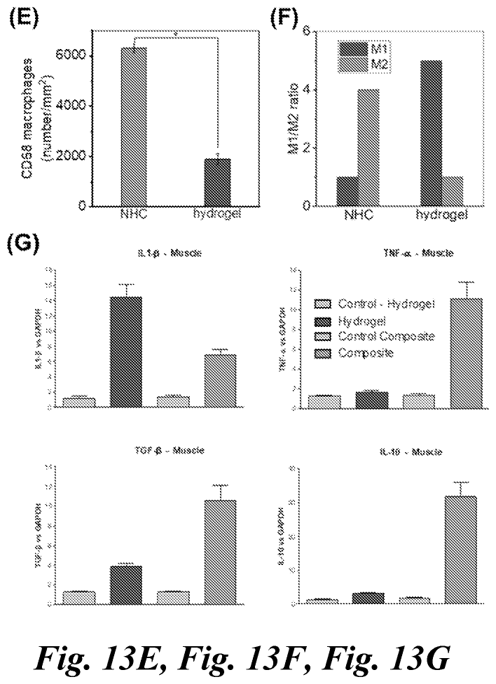

Hematoxylin & Eosin staining (FIG. 13A). To visualize the overall architecture of the injected NHC, hematoxylin and eosin staining of the target organ was performed. We used Hematoxylin 7211 and Eosin-Y, both from Thermo Scientific™ (Waltham, MA, USA), and followed the standard manufacture's protocol. SEM images were captured by FIBSEM microscopy from WSE the Materials Characterization and Processing Core (MCP).

Masson's Trichrome collagen staining (FIG. 13B and FIG. 13C). To study collagen deposition within the NHC and its hydrogel control, Masson's Trichrome staining was performed on 8 μm frozen tissue sections using the Stain Kit from Polysciences, Inc. (Warrington, PA) according to the manufacturer's instructions. Briefly, sections were initially incubated in Bouin's solution (overnight at room temperature). After washing with tap water for 1-2 min, the sections were stained with hematoxylin's working solution for 10 min, then washed with running tap water followed by distilled water. They were then incubated with Biebrich scarlet acid fuchsin solution for 5 min, phosphotungstic/phosphomolybdic acid for 10 min, aniline blue for 5 min, acetic acid (1%) for 1 min, and finally mounted on slides; distilled water rinses were performed in between each step. The sections were analyzed using polarized light microscopy under a 10× lens. For each specimen, 10-12 sections were taken from across the width of the muscle at the site of injection (approximately 20 μm apart) to quantify the amount of collagen staining. An image analysis system (Image J, NIH) was used to outline the injected material (NHC vs. hydrogel), and to calculate the percentage area of collagen staining. An average value, mean±standard deviation (SD), from the analyzed sections was taken to represent the collagen level for each muscle.

Macrophage immunohistochemical staining with CD68 (pan-marker), inducible nitric oxide synthase (iNOS, MI marker), and mannose receptor antibody (CD206, M2 marker) (FIG. 13D, FIG. 13E, and FIG. 13F). To quantify macrophage invasion of the injected material (NHC vs. hydrogel control) and determine their specific phenotype, immunohistochemical staining was performed on 8 μm frozen longitudinal tissue sections using the avidin-biotin-peroxidase complex (ABC) technique. Initially, the sections were treated with 3% H₂O₂ for 10 min to inhibit endogenous peroxidase activity. After rinsing in phosphate-buffered saline (PBS), the sections were incubated with 10% normal goat serum for 60 min: 0.1% TritonX-100 in PBS was added to the blocking buffer of iNOS and CD206 antibodies. The slides were then washed in PBS buffer, and incubated overnight in a humidified chamber at 4° C. with either a mouse anti-rat CD68 EDI antibody (Abcam, ab31630) at a dilution of 1:100 (in PBS containing 5% normal goat serum), or a rabbit Antirat iNOS (Abcam, ab15323) at a dilution of 1:100 (in 10% NGS+0.05% Tween-20), or a rabbit anti-rat CD206 (Abcam, ab64693) at a concentration of 0).5 ug/ml (in 10% NGS+0.05% Tween-20). Sixteen hours later, the sections were washed in PBS buffer and incubated for 1 h in a humidified chamber at room temperature with either a biotinylated goat anti-mouse IgG (Abcam, ab47844) diluted 1:300, or a biotinylated goat Anti-Rabbit (Abcam, ab6720) diluted 1:500 in PBS containing 5% normal goat serum. After further rinsing in PBS buffer, incubation was performed with either the peroxidase conjugated biotin-avidin complex (VECTASTAIN® Elite® ABC HRP Kit, PK-6100) or alkaline phosphatase conjugated biotin-avidin complex (VECTASTAIN® ABC-AP Staining KIT, AK-5000) for 30 min in the dark. Finally, the sections were washed in PBS buffer and incubated with either 3,30 diaminobenzidine (DAB, Vectorlabs, SK-4100) for 1 min or VECTOR Red Alkaline Phosphatase (SK-5100) for 25 min, rinsed in tap water, and finally counterstained with hematoxylin. Negative control sections were incubated using identical steps but omitting the primary antibody. The demonstration of positive cells in frozen sections of the spleen of the experimental animals was used as a positive control. For each specimen, 10-12 sections were taken from across the width of the injected material (approximately 20 μm apart) to quantify the number of macrophages. The number of positive cells was recorded in 6 random areas of the injected material, each consisting of 100 mm in width and extending 100 mm in length. All cell profiles visualized in a single plane of focus were counted. Counts are expressed as the number of positively stained cells per mm2 of tissue area. A mean±SD value was determined for each injection.

Inflammatory cytokines gene expression, qRT-PCR (FIG. 13G). To study the dynamics of pro- and anti-inflammatory cytokines in the injected muscle, mRNA expression of TNF-α, IL-1β, TGF-β and IL-10 was quantified using quantitative RT-PCR. Total RNA from snap-frozen muscle tissue was isolated using Trizol reagent (Life Technologies, Grand Island, NY), and then purified using the RNeasy mini kit (Qiagen, Valencia, CA) as previously described (17). The RNA integrity was evaluated with an Agilent 2100 Bioanalyzer (Agilent Technologies, Palo Alto, CA), and concentration was measured using a Nanodrop 8000 spectrophotometer (NanoDrop Products, Wilmington, DE). cDNA was generated using the Superscript III First Strand synthesis system for RT-PCR with Oligo(dT) primers, according to the manufacturer's instructions (Invitrogen, California, USA). qRT-PCR was performed with TaqMan probes and primers using QuantStudio™ 12 K Flex Real-Time PCR System (Life Technologies). Briefly, 1 mg of total RNA from each sample was reverse transcribed in a 20 ml reaction using SuperScript R: III First-Strand cDNA synthesis kit (Life Technologies). After optimized dilution of the resulting cDNA, PCR reaction was carried out in 20 ml reaction volumes containing diluted cDNA (20 ng RNA input) and gene-specific probes/primers as per manufacturer's protocol. An average threshold-cycle (Ct) from triplicate assays was used to determine the GAPDH-normalized gene expression.

2.3.3 Characterization of NP in vitro release in NHC and Bioactivity of Released IGF-1

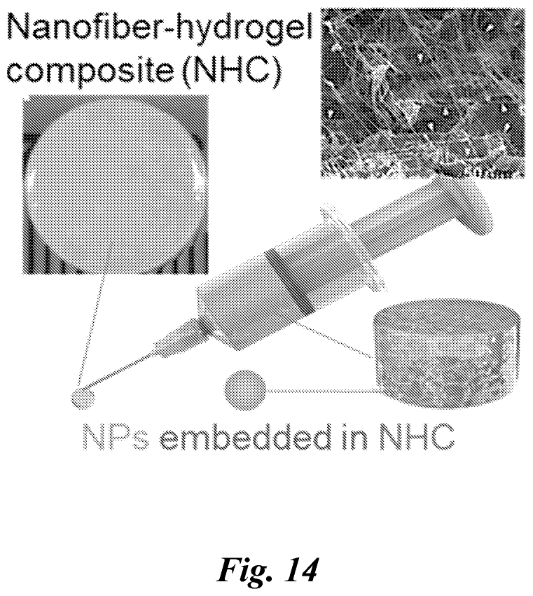

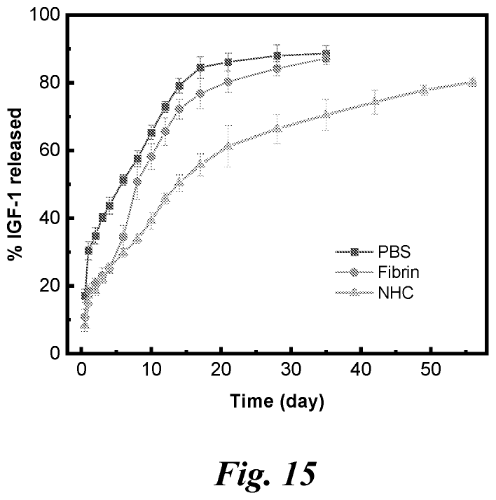

To evaluate the NP release embedded in NHC, the NP PBS solution was pre-mixed with HA solution prior to crosslinking. The homogeneity was shown in FIG. 14. The formed NHC with IGF-1 NP was placed in separate 50-mL centrifuge tubes with 10 mL and 1 mL PBS. At select times, medium was collected and replaced with PBS. The microBCA assay and a fluorescent plate reader measured IGF-1 concentration in medium from the dialysis tube and fibrin gel, respectively (FIG. 15).

Released IGF-1 samples were lyophilized and resuspended at a concentration series of 0.0001, 0.001, 0.01, 0.1, 1, 2, 5, 10, 50, 100 and 1000 ng/ml before being transferred to a 96-well plate. The bioactivity was tested against C2C12 myoblasts (ThermoFisher) and immortalized primary human SCs (courtesy of Dr. Ahmet Hoke, Department of Neurology, Johns Hopkins School of Medicine). C2C12 myoblasts were grown in a high glucose, no sodium pyruvate Dulbecco's modified Eagle's medium (DMEM; Gibco Life Technologies) supplemented with 20% fetal bovine serum (FBS; Sigma-Aldrich), 1% penicillin/streptomycin (Gibco Life Technologies), and 2 mM 1-glutamine at 37° C. and 5% CO2. The human SCs were cultured in a high glucose, no sodium pyruvate DMEM supplemented with 10% FBS, 0.2% glucose (Gibco Life Technologies), 1% penicillin/streptomycin, 2 mM l-glutamine, and 2 mM forskolin (Sigma-Aldrich) at 37° C. and 5% CO2. Each well of the 96-well plate was seeded with either 5000 C2C12 myoblasts or immortalized primary human SCs. After incubating with serially diluted IGF-1 samples for 24 h, 20 μL alamarBlue assay reagent (ThermoFisher) was added and incubated for 4 h before whole plate analysis using an absorbance plate reader at 570 nm (600 nm reference wavelength, Table 2).

TABLE 2
Bioactivity retention of released IGF-1 from NHC.

| | |
|---|---|
| | Relative Activity (%) tested in |
| Time point when release | either myoblasts or Schwann cells |

| samples were collected | Myoblasts | Schwann Cells |
|---|---|---|
| Day 0 (extracted from NPs) | 100 ± 3.7 | 100 ± 4.2 |
| Day 7 | 93.8 ± 3.2 | 91.5 ± 2.4 |
| Day 21 | 86.4 ± 4.1 | 89.7 ± 3.2 |
| Day 42 | 78.5 ± 2.6 | 81.3 ± 3.3 |

### 2.3.4 Animal Care and Use

Rats. 5-week-old to 6-week-old male Lewis rats (185-200 g, Charles Rivers Laboratories) were used for this study. All surgeries (including peri-operative care) followed the protocols of the Johns Hopkins University Animal Care and Use Committee (Protocol #RA18M74) according to the guidelines established by the National Institutes of Health and the American Association for the Accreditation of Laboratory Animal Care (NIH Publications #8023). All animals were kept in a central animal care facility with a 12-h light/12-h dark cycle and provided with food and water ad libitum. Surgical procedures and grip strength testing were conducted under general anesthesia using 2.5% inhaled isoflurane. All animals received subcutaneous buprenorphine (0.1 mg/kg) analgesia and enrofloxacin (1 mg/kg) prophylaxis postoperatively.

Macaques. Two adult male rhesus macaques (Macaca mulatta: age 12; weight 12-12.5 kg) were used in this study. Both macaques were singly housed and fed a standard commercial diet (2050 Teklad Gloval 20% Protein Primate Diet, Harlan Laboratories) 5 days per week with a range of food enrichment items. Water was freely available via an automatic watering system. This protocol was approved by the Institutional Animal Care and Use Committee of Johns Hopkins University (Protocol #PR19M423). All procedures were conducted according to the guidelines established by the National Institutes of Health and the American Association for the Accreditation of Laboratory Animal Care (NIH Publications #8023).

### 2.3.5 Animal Surgical Model

Rat chronic denervation model. A chronic median nerve denervation model in rats was chosen to assess the biocompatibility and immunogenicity of the NHC, to optimize the in vivo release kinetics of IGF-1 from the NHC NPs complex, to optimize the dosing regimen of NHC IGF-1 NPs injections, and to test the efficacy of this therapeutic strategy in improving functional outcomes. This is a pre-clinical nerve regeneration model known to recapitulate the challenges to human nerve regeneration. The median nerve is initially transected, and its distal stump sutured to the neighboring muscles. After a 12-week denervation period, the incision is re-opened, and the ulnar nerve is transferred to the denervated median nerve stump. This will allow regeneration of the ulnar nerve axons into the denervated median nerve segment to reinnervate the forearm flexors.

Macaque proximal nerve cut and repair model. To evaluate the translational feasibility of our treatment modality, we chose a proximal median nerve transection and repair model in macaques. An incision was made over the antebrachial area. The proximal median nerve was identified. It was transected, repaired, and then the NHC IGF-1 NPs (vs. normal saline control) was applied to the coaptation site, the exposed distal stump and injected into the forearm flexors. Injections were repeated every 6 weeks (as determined by our ELISA experiments). This model allowed us to serially measure the muscle IGF-1 concentration (through small punch biopsies of the flexor carpi radialis), and to assess the efficacy of the NHC IGF-1 NPs in improving functional outcomes.

2.3.6 Characterization of IGF-1 local distribution and retention in the target tissue

Confocal imaging of the target tissue. Serial confocal imaging of the injected muscle was performed to track the short and long-term localization of the NHC IGF-1 NPs complex to its target structure. The NHC was labeled with F8BT, the NP with Cy3 and IGF-1 with Cy5 fluorophores, respectively. Animals were sacrificed at different timepoints (2, 4, and 8 weeks). At each time point, animals (n=4) were sacrificed using deep anesthesia. Both the injected and contralateral structures was harvested. The contralateral non-injected nerve/muscle served as a negative control. The harvested tissue was fixed overnight in 4% PFA, and then placed into OCT molds, and flash frozen. Serial 10 μm sections were obtained and imaged using a confocal microscope (FIG. 16).

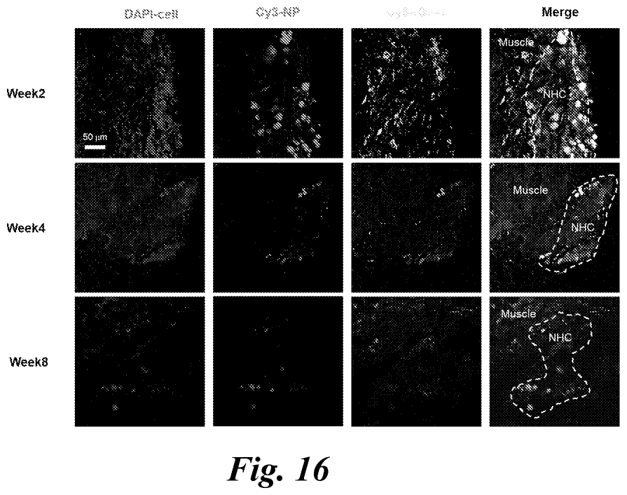

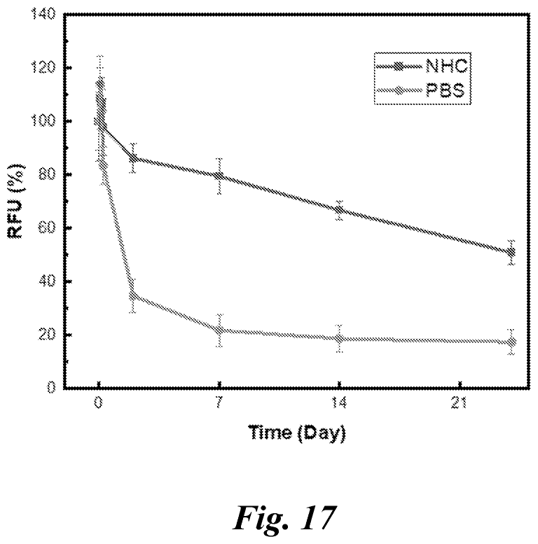

In Vivo bioluminescence imaging acquisition and analysis. In vivo bioluminescence imaging (BLI) was performed (using the Xenogen IVIS 200 system) to assess the efficacy of the NHC in maintaining the locally injected NPs and their protein content next to their target organ, and to look for any systemic dissemination of the payload. For the purpose of those experiments, we encapsulated the NPs with BSA (instead of IGF-1) as BSA labeling is easier and cheaper. The NPs were labeled with Cy5.5 fluorophores. The NHC loaded with the NPs was then injected into the forearm musculature or deposited around the median nerve repair site in rats. To quantify bioluminescence activities, an ROI was drawn in the region of the target muscle at each of the different time points. Automated measurement methods were used to identify bioluminescent emissions automatically within a defined threshold. The total flux of photons (or radiance) in each pixel (photons/s) was integrated over the ROI area and then multiplied by one steradian (sr). Relative ROI values were obtained and compared between the different groups (FIG. 17).

### 2.3.7 NHC Degradation and IGF-1 Retention in NHC in Rats

To assess the degradation kinetics of our scaffold, we injected a fixed weight of the NHC into the forelimb musculature of rats. We then sacrificed the rats, using deep anesthesia, at different timepoint, n=4 per timepoint. The injected muscle was harvested in its entirety (as one bloc). It was carefully opened and the remaining NHC material were dissected out, lyophilized and weighted. A mean±SD value of the weight of the remaining NHC material was determined at each timepoint.

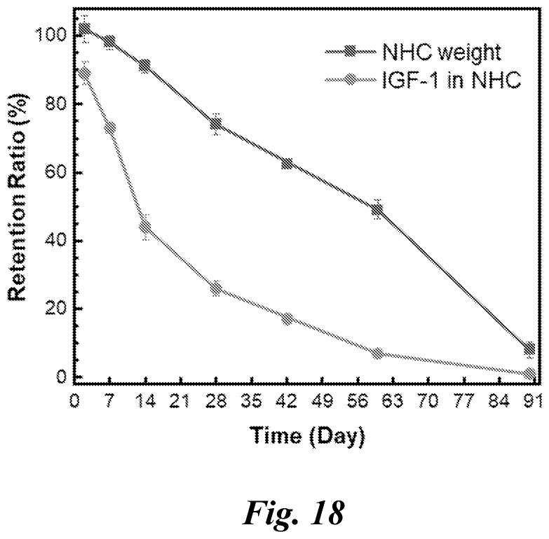

Also, IGF-1 concentration inside the NHC were then measured by extracting the IGF-1 out of the lyophilized materials using the same method described in Example 1. A mean±SD value of the weight of the remaining IGF-1 inside NHC material was determined at each timepoint (FIG. 18).

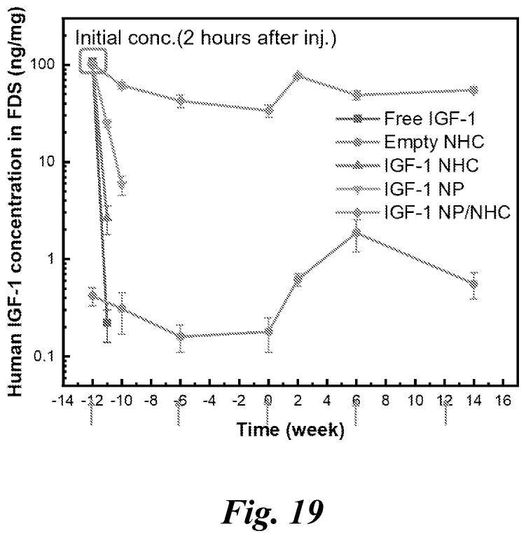

To assess the tissue residual IGF-1 concentration, we sacrificed the rats, using deep anesthesia, at different timepoint, n=4 per timepoint and harvested the target muscle (FDP, FDS). IGF-1 concentration was measured using the same method described in Example I (FIG. 19).

### 2.3.8 Functional Outcomes Measures in Rats

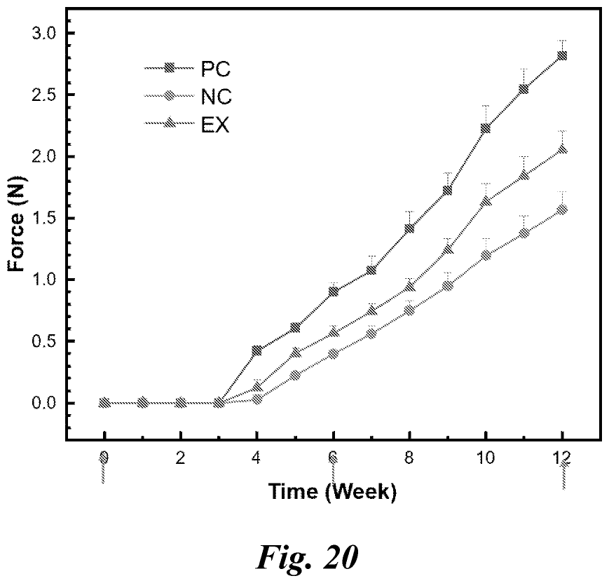

Grip strength testing. To track functional motor recovery over time, rats and macaques underwent serial stimulated grip strength testing (SGST), starting one week post nerve coaptation surgery for rats. SGST is a recently validated method that provides reliable measurements of functional motor recovery over time. It involves percutaneous stimulation of the median nerve to induce maximal tetanic contraction of the digital flexors. With the animal under general anesthesia, the forelimb is secured in 90° of abduction. A pair of needle-point electrode stimulators (ADInstruments) are placed 1 mm apart in the avilla for rats (using anatomical landmarks-just proximal to the insertion of the pectoralis major muscle onto the humerus). The proximal median nerve is then percutaneously, electrically stimulated supra-maximally at 10 V in order to ensure maximal tetanic contraction of the median-innervated flexors. Following induction of full digital flexion resulting in composite grasp, a metal loop fixed to an electronic force meter (Chatilon DE II, Ametek) is then placed on the palm of the animal and secured within the flexed digits and the force meter is distracted away from the animal until grasp is lost. The maximal force generated prior to loss of grasp (load to failure) is recorded in Newtons. Three trials were performed at both the 50 and 100 Hz frequencies, and the greater mean value between the two frequencies was used (FIG. 20).

### 2.3.9 Measurement of IGF-1 Concentration in Macaque

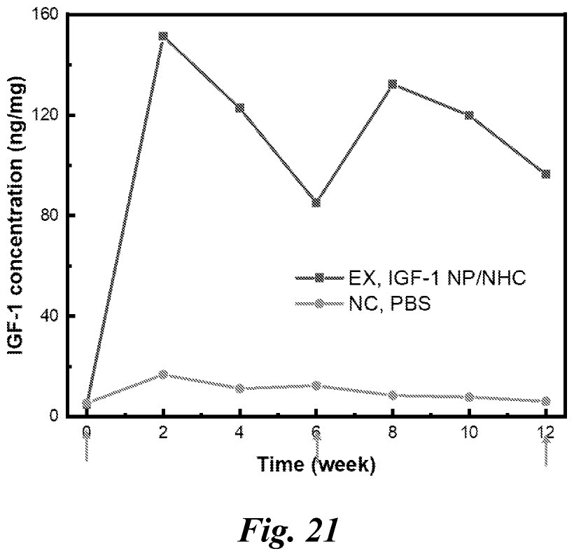

To measure serum IGF-1, 4 mL of blood drawn from macaque under deep anesthesia were taken from the dorsal tail vein and incubated at RT for 20 min before 10 min of 3000 rpm centrifugation at 4° C. Supernatant was flash frozen and stored at −80° C. To measure tissue IGF-1 in macaques, we performed serial flexor carpi radialis punch biopsies (every 2 weeks up to 12 weeks). 300 μL complete extraction buffer (100 mM Tris, pH 7.4, 150 mM NaCl, 1 mM egtazic acid, 1 mM ethylenediaminetetraacetic acid, 1% Triton X-100, 0.5% sodium deoxycholate, phosphatase inhibitor cocktail (1 mL, 50× stock), protease inhibitor cocktail (with phenyl methyl sulfonyl fluoride, Abcam, 250 μL, 500× stock) was added to each 5-mg piece of tissue before homogenization and shaking at 4° C. for 2 h. The suspensions were combined and centrifuged for 20 min at 13,000 rpm at 4° C. and placed on ice. The supernatant was aliquoted to a chilled tube for ELISA (Abcam) according to the manufacturer's protocols (FIG. 21).

### 2.3.10 Functional Outcomes Measures in Rats

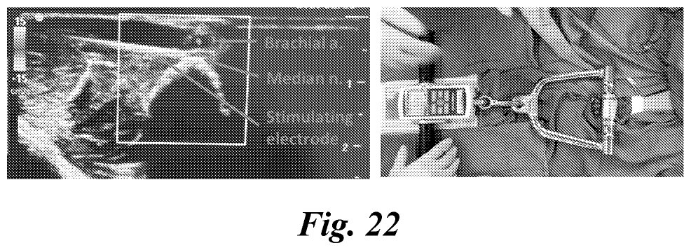

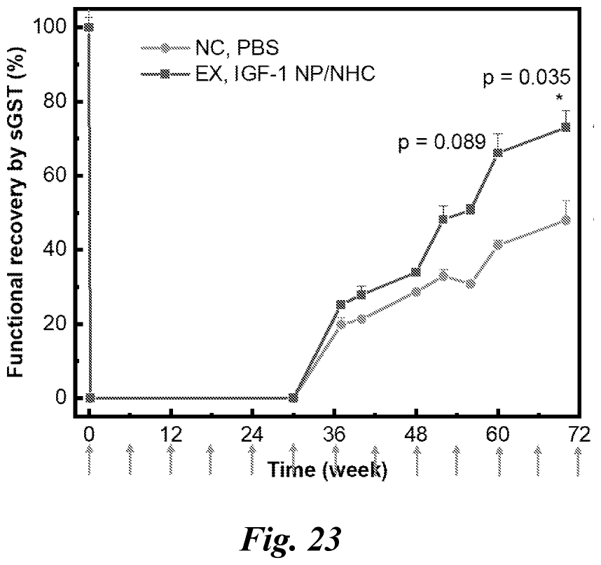

Grip strength testing. To track functional motor recovery over time, rats and macaques underwent serial stimulated grip strength testing (SGST), starting 30 weeks post nerve transection for macaques. SGST is a recently validated method that provides reliable measurements of functional motor recovery over time. It involves percutaneous stimulation of the median nerve to induce maximal tetanic contraction of the digital flexors. With the animal under general anesthesia, the forelimb is secured in 90° of abduction. A pair of needle-point electrode stimulators (ADInstruments) are placed 1 mm apart in the avilla for rats (using anatomical landmarks-just proximal to the insertion of the pectoralis major muscle onto the humerus). For macaques, we used ultrasound guidance to air in placement of the stimulating electrodes (FIG. 22). The proximal median nerve is then percutaneously, electrically stimulated supra-maximally at 10 V in order to ensure maximal tetanic contraction of the median-innervated flexors. Following induction of full digital flexion resulting in composite grasp, a metal loop fixed to an electronic force meter (Chatilon DE II, Ametek) is then placed on the palm of the animal and secured within the flexed digits and the force meter is distracted away from the animal until grasp is lost. The maximal force generated prior to loss of grasp (load to failure) is recorded in Newtons. Three trials were performed at 100 Hz frequencies (FIG. 23).

### 2.3.11 Statistical Analysis

Non-parametric tests were used throughout the study. A Mann-Whitney test was used to compare two independent groups. For multiple groups' comparison (e.g., bioluminescence slopes), the Kruskal Wallis one-way analysis of variance by ranks was used. Provided significant differences were detected, the Dwass-Steel-Chritchlow-Fligner post hoc analysis method was applied for pairwise comparisons. As for the SGST, differences in muscle force recovery over time between the groups was tested using a nonparametric analysis of longitudinal data. Values are expressed as mean±SD. A p value of <0.05 was considered significant. All analyses were performed using SPSS Statistics version 23 (IBM, Armonk, NY, USA), as well as the R package “nparLD”.

### 2.4 Results and Discussion

### 2.4.1. Characterization of NHC-mediated Pro-Regenerative Response

To investigate the NHC-mediated host cellular response in vivo, we injected the forearm musculature of Lewis rats with either NHC or hydrogel control.

Hematoxylin and eosin staining (FIG. 13A). The overall architecture of the muscle was preserved in the NHC group. We noted that the NHC contained a large number of inflammatory cells.

Masson's Trichrome staining (FIG. 13B and FIG. 13C). The anti-inflammatory M2 macrophages led to decreased collagen deposition within the NHC as compared to hydrogel (7.98%+0.56 vs. 21.63%+1.37, p<0.05). This has proved that the NHC provided a regenerative environment with no obvious capsule formation, facilitating the IGF-1 release out of the NHC.

CD68 (pan-macrophage marker) immunohistochemistry (FIG. 13D and FIG. 13E). The predominance of macrophages within the NHC was confirmed with CD68 immunohistochemical staining. The hydrogel, on the other hand, had a much smaller number of CD68 positive cells (6,300/mm²+454 vs. 1,900/mm²+211, respectively, p<0.05).

Macrophage polarization (FIG. 13D and FIG. 13F). Further phenotypic characterization of the invading macrophages was performed using CD68/M1 and CD68/M2 co-staining. This revealed that most of the macrophages within the NHC were of the anti-inflammatory pro-regenerative M2 phenotype (M1:M2 ratio, 1:4) whereas those present within the hydrogel were of the pro-inflammatory MI phenotype (M1:M2 ratio, 5:1).

qRT-PCR (FIG. 13G). Gene expression of pro- and anti-inflammatory cytokines within the injected forearm musculature and the nerve was assessed. Muscle analysis showed a significant increase in the expression of the anti-inflammatory cytokines IL-10 and TGF-β in the NHC group as compared to the hydrogel group (p<0.01). It also showed a significant increase of the pro-inflammatory cytokine IL-Iβ in the hydrogel group as compared to NHC (p<0.05). TNF-α expression was significantly increased in the NHC group (p<0.01). As for the nerve analysis, no significant difference in gene expression was identified among those inflammatory cytokines. Of note, only the nerve segment (without its surrounding tissue environment) was analyzed. Our analysis of the nerve inflammatory response could have been more accurate had it included some of the surrounding tissue.

### 2.4.2. Evaluation of In Vivo Release and Efficacy

The IGF-1 NP has been pre-mixed with HA-Ac and electrospun PCL nanofibers. The mixing solution was vortexed to ensure sufficient mixing. Crosslinker was then added to form the NHC and IGF-1 NP was loaded inside the NHC matrix (FIG. 14). Incorporated into the NHC, more than 70% of IGF-1 has been released out at week 6 and almost 80% at week 8. NHC extended up to two months of sustained release before plateau (FIG. 15). The bioactivity of released IGF-1 measured by in vitro cell proliferation assay has been determined to be 78.5% for C2C12 myoblasts and 81.3% for human Schwann cells at week 6 (Table. 2).

### 2.4.3. IGF-1 In Vivo Release and Retention

We used Cy3-labeled NPs (red) and Cy5-labeled IGF-1 (cyan) to formulate the IGF-1 NP and injected in rats to assess the retention of NP and IGF-1 at the injection site.

IGF-1 being released into muscle from NHC for at least 8 weeks (FIG. 16). Cells (DAPI, blue) infiltrated into the NHC within 2 weeks. IGF-1 has been released out of the NP/NHC carrier and transported across the host-tissue interface that delivered into the muscle. The released IGF-1 signal has been decaying as time elapsed. The NHC could release 56% of IGF-1 at week 2 and 78% of IGF-1 at week 4. There was only less than 10% of total IGF-1 left inside the NHC at week 8 (FIG. 18).

NHC enhanced NP local retention (FIG. 17). Fluorescent intensity of NP has been recorded using in vivo imaging system (IVIS). NPs using PBS as carrier lost more than 60% of signals within 2 days and 80% for 7 days. NPs using NHC as carrier could maintain more than 60% of signals after 3 weeks at a slowly decaying manner. NHC itself degraded at a linear rate for 3 months (FIG. 18).

IGF-1 NP NHC maintained tissue IGF-1 level (FIG. 19). Free IGF-1 with no carrier could not maintain tissue IGF-1 level for more than a few days. IGF-1 NP without NHC or IGF-1 in NHC without NP both could not sustained the IGF-1 concentration in muscle for more than 2 weeks. NP and NHC were both critical in maintaining IGF-1 locally. IGF-1 NP/NHC could keep the IGF-1 concentrations above therapeutic levels when redosing at an every 6-week manner. The IGF-1 NP/NHC group shown more than 100 folds of IGF-1 concentration as compared to the negative control group. Same trend has been reproduced in the macaque trials (FIG. 21).

### 2.4.4. IGF-1 NPs Maintained SC Proliferation During Denervation

Grip strength motor recovery is the primary outcome of interest as it recapitulates the ultimate clinical goal of nerve repair. We measured grip strength weekly for rats, throughout the 12-week regeneration period, using a validated method that reliably tracks functional recovery over time. We had a total of 3 groups (n=8 per group). The positive control group consisted of an immediate ulnar to median nerve transfer without a denervation period. The experimental group underwent a 12-week denervation period followed by the nerve transfer. In addition, those animals received injections of the NHC IGF-1 NPs system, every 6 weeks, into the target muscle and next to the nerve. The negative control group was identical to the experimental group except that the injections consisted of empty NHC.

Motor recovery was first detected at a median of 4 weeks in all groups (p>0.05), suggesting that IGF-1 treatment did not significantly accelerate initial reinnervation (FIG. 20). This is most likely due to the short regenerative distance from the nerve repair site to the most proximal target muscle branches. However, a stronger grip strength was recovered in the IGF-1 treated animals (as compared to negative control) starting week 6. This difference persisted through the whole measurement process. The positive control group had the strongest and fastest functional recovery.

In a pilot study using a macaque model, we dissected and repaired the nerve immediately, and no motor functional recovery has been observed for the first 30 weeks due to longer distance for regenerating nerve to travel in macaques before reinnervating into target muscles. The experimental group ended with 76% recovery versus 45% in the negative control group at week 70 (FIG. 23). Both the rat and macaque models have shown an improved recovery for about 30% in terms of grip strength.

### 2.5 Summary

The quest for a clinically translatable therapeutic strategy to improve outcomes following peripheral nerve repair has intensified with the recent advancements in tissue engineering. The ultimate goal is to create an injectable biomaterial scaffold that incorporates specific biochemical and topographical cues to induce a long-lasting regenerative micro-environment capable of sustaining the lengthy axonal growth process while limiting denervation-induced muscle and SCs atrophy. Local delivery of IGF-1 constitutes a promising biochemical signal with well-described trophic and anti-apoptotic effects on neurons, myocytes, and SCs. Our group has successfully encapsulated IGF-1 into biodegradable and biocompatible PEG-b-PCL NPs. This encapsulation allowed a sustained release of bioactive IGF-1 to the regenerating nerve and its target muscle for two weeks in a chronic denervation animal model. However, a carrier that localizes the NPs next to their target organ for an extended period (longer than two weeks) while still enabling a controlled release of IGF-1 was needed to achieve clinical translation of this novel growth factor-based treatment strategy.

The NHC can be further functionalized with small protein therapeutics to behave as an injectable drug delivery system with clinically favorable release kinetics. We homogeneously loaded the NHC with IGF-1 NPs to provide a sustained release of bioactive IGF-1 for a total duration of 6 weeks in local tissues with enhanced retention. The NHC injections proved to be easily applicable in both rodent and large animal models of PNI.

### Example 3

Sustained Delivery of Agrin from Nanoparticles Improves Neuromuscular Junction Reinnervation and Functional Recovery After Chronic Denervation

### 3.1 Overview

Peripheral nerve injuries (PNI) currently affect more than one million people worldwide, and the occurrence of trauma-induced PNI is steadily on the rise. PNI are notoriously devastating and life-altering as patients can face severe lifelong disabilities including sensory loss, motor loss, and neuropathic pain. Despite advancements in microsurgical techniques and basic and translational research, traditional treatments continue to have unsatisfactory clinical outcomes.

Among the many factors that influence peripheral nerve injury prognosis, including age and co-morbidities, the amount of time that elapses prior to end-organ reinnervation is the most consequential. This is evidenced by the poor outcomes that tend to occur with proximal nerve injuries and delayed repairs. Denervation results in several progressive degenerative processes that impede regeneration and minimize the degree of functional return achievable on reinnervation. One such process involves atrophy of denervated muscle. Without neural input, skeletal muscle loses bulk and fibrosis, with myofibrils dwindling in size and number. As muscle atrophy progresses, the amount of contractile force possible on reinnervation is permanently diminished. Further hindering outcomes, chronically denervated neuromuscular junctions (NMJs) within the denervated muscle degrade and lose their capacity to receive regenerating axons.

Given the importance of prompt reinnervation, much attention has been directed towards developing therapies to accelerate axonal regeneration beyond the typical rate of 1 mm/day, and a number of experimental agents have demonstrated efficacy in this regard. However, the ideal therapy would also act to maintain muscle receptivity to reinnervation during the process of regeneration. Growth factor-based therapies have emerged as promising therapeutics due to their stimulatory actions in a number of cell types, including neurons, myocytes, and Schwann cells.

Translational studies in rodents performed by our group and others have demonstrated the efficacy of growth hormone and its derivative IGF-1 in improving nerve regeneration, muscle integrity, and functional recovery. However, further exploration of regenerative mechanisms and therapeutics is necessary as there remains area for improvement within functional and histological outcomes after IGF-1 therapy. This is likely due to the substantial percentage of NMJs that remain denervated despite therapeutic intervention. Following denervation, acetylcholine receptors (AChRs) within NMJs dramatically destabilize and acquire a reduced half-life from 10 days to a mere 2-3 days. This destabilization and increased receptor turnover reduces synaptic strength and consequently impairs receptivity to reinnervation. An approach that prolongs NMJ viability until reinnervation can occur would likely improve functional recovery compared to other current therapeutic modalities.

Agrin, a proteoglycan released by motor nerve terminals into the synaptic cleft, is essential for the stabilization of AChR aggregates as well as the formation and stabilization of NMJs. Previous studies have found that local delivery of agrin following denervation serves to transiently improve NMJ morphology. Given the short half-life of denervated AChRs and the lengthy time required for regeneration, it is thought that providing a sustained, local delivery of agrin will stabilize denervated NMJs until regenerating axons reach them, thereby promoting reinnervation and consequently improving functional recovery.

### 3.2 Background

One of the most critical factors contributing to poor outcomes result is the prolonged period of latency prior to reinnervation. Over time, the absence of muscle innervation causes myofibril shrinkage, neuromuscular junction degradation, and ultimately irreversible atrophy that limits meaningful functional motor recovery. Given the importance of prompt reinnervation, much attention has been directed towards developing therapies to accelerate axonal regeneration and decrease the time to reinnervation. Although a number of experimental agents have demonstrated efficacy in this regard, there remains area for improvement in functional and histological outcomes. This is likely due to the substantial percentage of neuromuscular junctions (NMJs) that remain denervated despite therapeutic intervention. Acetylcholine receptors (AChRs) within denervated muscle rapidly destabilize and consequently degrade NMJs, thereby limiting meaningful functional motor recovery. We seek to further improve functional outcomes by evaluating a therapeutic that prolongs NMJ viability until reinnervation can occur.

We developed biodegradable nanoparticles to encapsulate agrin, a proteoglycan involved in the formation and stabilization of NMJs. Previous studies by our group and others have found that the use of agrin as a therapeutic after denervation serves to transiently reduce NMJ degradation. Given the lengthy time required for regeneration, we incorporated the nanoparticle-encapsulated agrin into a nanofiber hydrogel composite carrier to allow for sustained, local delivery of agrin to target tissues.

### 3.3 Methods

### 3.3.1 NP Fabrication.

Agrin was first complexed with dextran sulfate to create polyelectrolyte complex (PEC) cores by FNC, which were then encapsulated in biodegradable amphiphilic block co-polymers to form the NPs by FNP. Varying ratios of PEC:polymer were evaluated to maximize loading efficiency and release kinetics. In vitro NP release kinetics were evaluated and mitogenic activity of released agrin was compared to native agrin.

### 3.3.2 Tissue Harvest for NMJ Quantification.

The effects of locally-delivered agrin-NPs on denervated muscle were assessed in a rat tibial nerve transection-without-repair model. Lewis-Norway rats were injected with low, medium, or high doses of agrin-NPs incorporated into a nanofiber-hyaluronic acid hydrogel composite (NHC) gel, empty-NPs within NHC, free agrin, or saline. After 6 weeks, animals were sacrificed and the soleus, lateral and medial gastrocnemius muscles were harvested for analysis.

### 3.3.3 In Vivo Functional Test.

Following nerve transfer, all animals underwent 16 weeks of regeneration and grip strength assessment. Denervated animals were injected with agrin-NP/NHC, free agrin, or empty-NP/NHC every six weeks from time of median nerve injury. A total of five injections were given: 1) at the time of median nerve injury, 2) after 6 weeks of denervation, 3) at the time of nerve transfer after 12 weeks of denervation, 4) week 6 post nerve transfer, 5) week 12 post nerve transfer. The “no denervation” control group only received the ulnar-to-median nerve transfer and saline injections.

The detailed methods to make agrin NP, to load NP in NHC, to evaluate in vitro release kinetics, to assess the NMJ morphology and counts, to run tissue ELISA samples and to do grip strength tests were all using the methods described in Example 1 and 2.

### 3.4 Results and Discussion

### 3.4.1. Characterization of Agrin NP

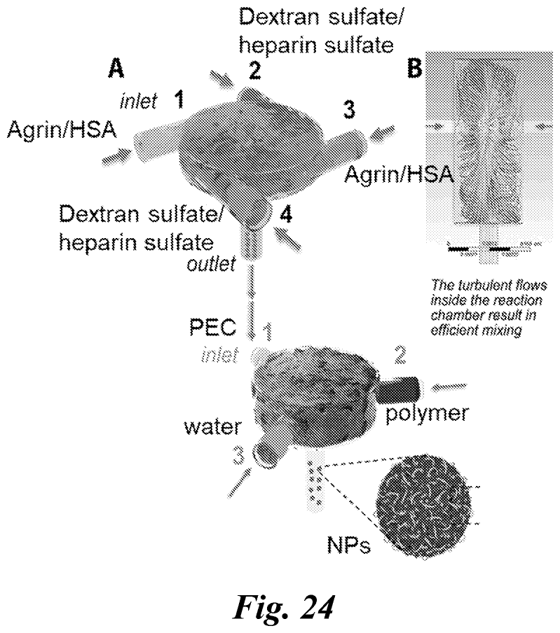

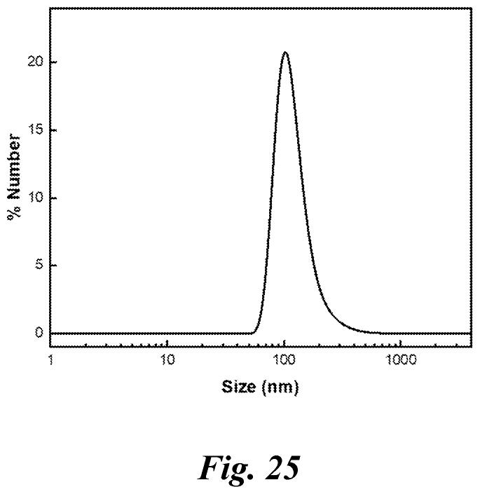

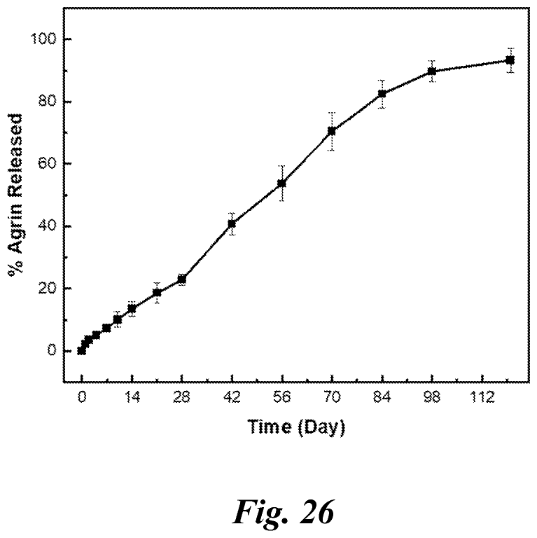

Agrin NP produced using the FNC/FNP in-tandem method (FIG. 24) had a narrow distribution by DLS (hydrodynamic size: 102 nm, PDI: 0.17, FIG. 25). As a large protein (200 kDa), agrin released ˜1% per day from NP/NHC for the first 12 weeks. Near-zero-order release profile has been achieved (FIG. 26).

### 3.4.2. Agrin NP NHC Preserved NMJ Structural Integrity

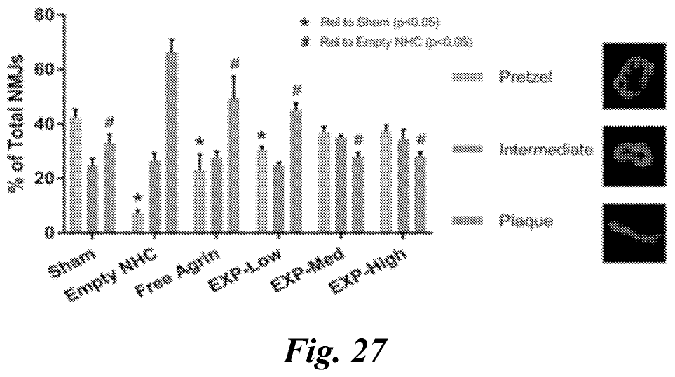

Agrin-NP treated animals retained significantly greater NMJ pretzel-like morphology after 6 weeks of denervation compared to free agrin and empty-NP groups with optimal benefit achieved by the medium dose (35.0% vs 23.1% free agrin, 35.0% vs 7.1% empty-NP; p<0.0001). Furthermore, both medium and high dose Agrin-NP treated animals demonstrated significantly lower NMJ plaque-like morphology than free agrin treated animals (p<0.0001). NMJ morphology of medium dose Agrin-NP treated animals were not significantly different than sham animals, suggesting optimal benefit was achieved at the medium dose (FIG. 27).

### 3.4.3. Agrin In Vivo Concentrations by ELISA

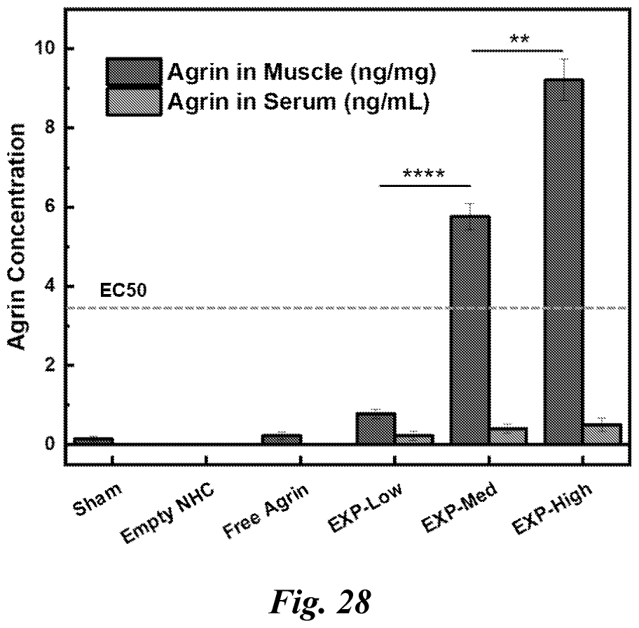

All Agrin-NP treated animals retained greater agrin levels in the soleus muscle as compared to free agrin-treated animals and endogenous agrin levels in sham animals at 6 weeks (p<0.001). Agrin levels were undetectable in serum at all agrin-NP doses. No significant differences were seen in myofibril cross-sectional area between Agrin-NP, free agrin, and empty-NP groups. No foreign body response was detected in empty-NP or Agrin-NP treated animals (FIG. 28). Agrin-NP/NHC treated animals demonstrated significantly greater NMJ reinnervation than free agrin (78.9% vs 66.8%, p<0.05) and empty-NP/NHC groups (78.9% vs 39.2%, p<0.001).

### 3.4.4. Agrin in NP NHC Improved Functional Outcomes (Grip Strength)

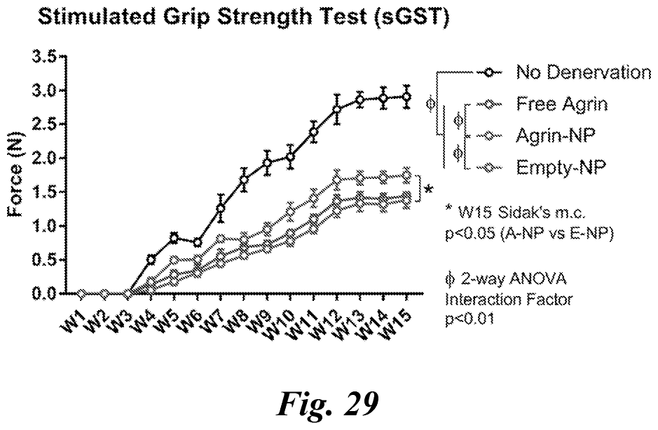

Agrin-NP/NHC treated animals exhibited significantly increased functional recovery throughout the regeneration period compared to free agrin (p<0.01 2-way ANOVA) and empty-NP/NHC treated animals (p<0.01 2-way ANOVA) At Week 15 (FIG. 29). Agrin-NP/NHC treated animals demonstrated an increase of 21.2% compared to free agrin (p=0.056) and of 26.8% compared to empty-NP/NHC animals (p<0.05).

### 3.5 Summary

Encapsulation of bioactive agrin with sustained release for over 70 days was achieved. Agrin-NP treatment in vivo limits neuromuscular junction degradation during denervation and thereby has potential as a therapeutic target to improve motor functional recovery. Agrin-NP/NHC treatment in vivo promotes neuromuscular junction reinnervation and improves functional recovery of forelimb grip strength. In the setting of denervation, sustained, localized delivery of agrin through the FNC/FNP delivery system prevented neuromuscular junction degradation during the regenerative period, resulting in increased reinnervation and improved functionality after peripheral nerve injury in a rodent model.

With previously established benefits of insulin-like growth factor 1 (IGF-1) nanoparticles on ameliorating the effects of chronic denervation, to combine Agrin-NPs and IGF-1-NPs and evaluate their hypothesized synergistic benefits would also empower the technology.

### REFERENCES

All publications, patent applications, patents, and other references mentioned in the specification are indicative of the level of those skilled in the art to which the presently disclosed subject matter pertains. All publications, patent applications, patents, and other references are herein incorporated by reference to the same extent as if each individual publication, patent application, patent, and other reference was specifically and individually indicated to be incorporated by reference. It will be understood that, although a number of patent applications, patents, and other references are referred to herein, such reference does not constitute an admission that any of these documents forms part of the common general knowledge in the art.
C. A. Taylor, D. Braza, J. B. Rice, T. Dillingham, The incidence of peripheral nerve injury in extremity trauma. Am J Phys Med Rehabil 87, 381-385 (2008).
C. N. Bruyns et al., Predictors for return to work in patients with median and ulnar nerve injuries. The Journal of hand surgery 28, 28-34 (2003).
B. M. Carlson, L. Billington, J. Faulkner, Studies on the regenerative recovery of long-term denervated muscle in rats. Restor Neurol Neurosci 10, 77-84 (1996).
D. Grinsell, C. P. Keating, Peripheral nerve reconstruction after injury: a review of clinical and experimental therapies. Biomed Res Int 2014, 698256 (2014).
O. A. Sulaiman, T. Gordon, Effects of short- and long-term Schwann cell denervation on peripheral nerve regeneration, myelination, and size. Glia 32, 234-246 (2000).
T. Gordon, N. Tyreman, M. A. Raji, The basis for diminished functional recovery after delayed peripheral nerve repair. J Neurosci 31, 5325-5334 (2011).
K. M. Chan, T. Gordon, D. W. Zochodne, H. A. Power, Improving peripheral nerve regeneration: from molecular mechanisms to potential therapeutic targets. Experimental neurology 261, 826-835 (2014).
E. L. Feldman, K. A. Sullivan, B. Kim, J. W. Russell, Insulin-like growth factors regulate neuronal differentiation and survival. Neurobiol Dis 4, 201-214 (1997).
M. Kanje, A. Skottner, J. Sjoberg, G. Lundborg, Insulin-like growth factor I (IGF-I) stimulates regeneration of the rat sciatic nerve. Brain Res 486, 396-398 (1989).
P. J. Apel et al., Effect of locally delivered IGF-1 on nerve regeneration during aging: an experimental study in rats. Muscle Nerve 41, 335-341 (2010).
D. N. Ishii, G. W. Glazner, S. F. Pu, Role of insulin-like growth factors in peripheral nerve regeneration. Pharmacol Ther 62, 125-144 (1994).
T. Shavlakadze, J. D. White, M. Davies, J. F. Hoh, M. D. Grounds, Insulin-like growth factor I slows the rate of denervation induced skeletal muscle atrophy. Neuromuscul Disord 15, 139-146 (2005).
T. N. Stitt et al., The IGF-1/PI3K/Akt pathway prevents expression of muscle atrophy-induced ubiquitin ligases by inhibiting FOXO transcription factors. Mol Cell 14, 395-403 (2004).
S. C. Bodine et al., Akt/mTOR pathway is a crucial regulator of skeletal muscle hypertrophy and can prevent muscle atrophy in vivo. Nat Cell Biol 3, 1014-1019 (2001).
M. F. Heszele, S. R. Price, Insulin-like growth factor I: the yin and yang of muscle atrophy. Endocrinology 145, 4803-4805 (2004).
C. L. Delaney, J. W. Russell, H. L. Cheng, E. L. Feldman, Insulin-like growth factor-I and over-expression of Bcl-xL prevent glucose-mediated apoptosis in Schwann cells. J Neuropathol Exp Neurol 60, 147-160 (2001).
M. Schumacher, I. Jung-Testas, P. Robel, E. E. Baulieu, Insulin-like growth factor I: a mitogen for rat Schwann cells in the presence of elevated levels of cyclic AMP. Glia 8, 232-240 (1993).
L. Yan et al., Anti-Apoptotic Effect of IGF1 on Schwann Exposed to Hyperglycemia is Mediated by Neuritin, a Novel Neurotrophic Factor. Mol Neurobiol 55, 495-505 (2018).
H. L. Cheng, M. Steinway, C. L. Delaney, T. F. Franke, E. L. Feldman, IGF-I promotes Schwann cell motility and survival via activation of Akt. Mol Cell Endocrinol 170, 211-215 (2000).
S. H. Tuffaha et al., Therapeutic augmentation of the growth hormone axis to improve outcomes following peripheral nerve injury. Expert Opin Ther Targets 20, 1259-1265 (2016a).
D. N. Ishii, G. W. Glazner, L. R. Whalen, Regulation of peripheral nerve regeneration by insulin-like growth factors. Ann N Y Acad Sci 692, 172-182 (1993).
S. H. Tuffaha et al., Growth Hormone Therapy Accelerates Axonal Regeneration, Promotes Motor Reinnervation, and Reduces Muscle Atrophy following Peripheral Nerve Injury. Plast Reconstr Surg 137, 1771-1780 (2016b).
V. E. Bianchi, V. Locatelli, L. Rizzi, Neurotrophic and Neuroregenerative Effects of GH/IGF1. Int J Mol Sci 18 (2017).
J. Saceda et al., Effect of recombinant human growth hormone on peripheral nerve regeneration: experimental work on the ulnar nerve of the rat. Neurosci Lett 504, 146-150 (2011).
J. Lopez et al., Growth Hormone Improves Nerve Regeneration, Muscle Re-innervation, and Functional Outcomes After Chronic Denervation Injury. Sci Rep 9, 3117 (2019).
J. Sjoberg, M. Kanje, Insulin-like growth factor (IGF-1) as a stimulator of regeneration in the freeze-injured rat sciatic nerve. Brain Res 485, 102-108 (1989).
M. Kanje, A. Skottner, G. Lundborg, J. Sjoberg, Does insulin-like growth factor I (IGF-1) trigger the cell body reaction in the rat sciatic nerve? Brain Res 563, 285-287 (1991).
E. Emel et al., Effects of insulin-like growth factor-I and platelet-rich plasma on sciatic nerve crush injury in a rat model. J Neurosurg 114, 522-528 (2011).
D. A. Tiangco, K. C. Papakonstantinou, K. A. Mullinax, J. K. Terzis, IGF-I and end-to-side nerve repair: a dose-response study. J Reconstr Microsurg 17, 247-256 (2001).
H. Fansa, W. Schneider, G. Wolf, G. Keilhoff, Influence of insulin-like growth factor-I (IGF-I) on nerve autografts and tissue-engineered nerve grafts. Muscle Nerve 26, 87-93 (2002).
G. Tae, M. Scatena, P. S. Stayton, A. S. Hoffman, PEG-cross-linked heparin is an affinity hydrogel for sustained release of vascular endothelial growth factor. Journal of Biomaterials Science, Polymer Edition 17, 187-197 (2006).
D. Wang, X. Yang, Q. Liu, L. Yu, J. Ding, Enzymatically cross-linked hydrogels based on a linear poly(ethylene glycol) analogue for controlled protein release and 3D cell culture. Journal of Materials Chemistry B 6, 6067-6079 (2018).
L. Tang et al., Enhancing T cell therapy through TCR-signaling-responsive nanoparticle drug delivery. Nature biotechnology 36, 707-716 (2018).
Z. He et al., Sustained release of exendin-4 from tannic acid/Fe (III) nanoparticles prolongs blood glycemic control in a mouse model of type II diabetes. Journal of Controlled Release 301, 119-128 (2019).
R. Xu et al., High strength astringent hydrogels using protein as the building block for physically cross-linked multi-network. ACS applied materials & interfaces 10, 7593-7601 (2017).
J. Majumder, O. Taratula, T. Minko, Nanocarrier-based systems for targeted and site specific therapeutic delivery. Advanced drug delivery reviews 144, 57-77 (2019).
Y.-P. Li et al., PEGylated PLGA nanoparticles as protein carriers: synthesis, preparation and biodistribution in rats. Journal of controlled release 71, 203-211 (2001).
W. Park, D. Kim, H. C. Kang, Y. H. Bae, K. Na, Multi-arm histidine copolymer for controlled release of insulin from poly(lactide-co-glycolide) microsphere. Biomaterials 33, 8848-8857 (2012).
B. K. Johnson, R. K. Prud'homme, Flash NanoPrecipitation of Organic Actives and Block Copolymers using a Confined Impinging Jets Mixer. Australian Journal of Chemistry 56, 1021-1024 (2003).
C. E. Markwalter, R. K. Prud'homme, Design of a small-scale multi-inlet vortex mixer for scalable nanoparticle production and application to the encapsulation of biologics by inverse flash nanoprecipitation. Journal of pharmaceutical sciences 107, 2465-2471 (2018).
C. E. Markwalter et al., Polymeric Nanocarrier Formulations of Biologics Using Inverse Flash NanoPrecipitation. The AAPS journal 22, 1-16 (2020).
Z. He et al., Size-controlled lipid nanoparticle production using turbulent mixing to enhance oral DNA delivery. Acta Biomaterialia 81, 195-207 (2018).
Z. He et al., Sustained release of exendin-4 from tannic acid/Fe (III) nanoparticles prolongs blood glycemic control in a mouse model of type II diabetes. Journal of controlled release: official journal of the Controlled Release Society 301, 119-128 (2019).
R. Hunter, I. Hers, Insulin/IGF-1 hybrid receptor expression on human platelets; consequences for the effect of insulin on platelet function. Journal of Thrombosis and Haemostasis 7, 2123-2130 (2009).
P. J. Hanwright et al., Stimulated grip strength measurement: validation of a novel method for functional assessment. Muscle & nerve 60, 437-442 (2019).
G. W. Glazner, D. N. Ishii, Insulin-like growth factor gene expression in rat muscle during reinnervation. Muscle Nerve 18, 1433-1442 (1995).
H. J. Stewart et al., Regulation of rat Schwann cell Po expression and DNA synthesis by insulin-like growth factors in vitro. Eur J Neurosci 8, 553-564 (1996).
A. Fex Svenningsen, M. Kanje, Insulin and the insulin-like growth factors I and II are mitogenic to cultured rat sciatic nerve segments and stimulate [3H]thymidine incorporation through their respective receptors. Glia 18, 68-72 (1996).
P. Caroni, P. Grandes, Nerve sprouting in innervated adult skeletal muscle induced by exposure to elevated levels of insulin-like growth factors. The Journal of cell biology 110, 1307-1317 (1990).
T. M. Raimondo et al., Combined delivery of VEGF and IGF-1 promotes functional innervation in mice and improves muscle transplantation in rabbits. Biomaterials 216, 119246 (2019).
K. J. Livak, T. D. Schmittgen, Analysis of relative gene expression data using real-time quantitative PCR and the 2 (-Delta C (T)) Method. Methods 25, 402-408 (2001).
Y. Wen et al., MyoVision: software for automated high-content analysis of skeletal muscle immunohistochemistry. J Appl Physiol (1985) 124, 40-51 (2018).
B. Kern et al., A Novel Rodent Orthotopic Forelimb Transplantation Model That Allows for Reliable Assessment of Functional Recovery Resulting From Nerve Regeneration. Am J Transplant 17, 622-634 (2017).
O. A. Sulaiman, T. Gordon, Role of chronic Schwann cell denervation in poor functional recovery after nerve injuries and experimental strategies to combat it. Neurosurgery 65, A105-114 (2009).

Although the foregoing subject matter has been described in some detail by way of illustration and example for purposes of clarity of understanding, it will be understood by those skilled in the art that certain changes and modifications can be practiced within the scope of the appended claims.

That which is claimed:

1. A method for preparing one or more nanoparticles comprising an amphiphilic block copolymer having a polyelectrolyte complex comprising one or more therapeutic small proteins and a counter ion polymer encapsulated therein, the method comprising:
(a) assembling the polyelectrolyte complex comprising a therapeutic small protein and a counterion polymer, wherein the counter ion polymer has a charge enabling it to bind electrostatically to the one or more therapeutic small proteins; and
(b) encapsulating the polyelectrolyte complex with an amphiphilic block copolymer through a flash nanoprecipitation (FNP) process.

2. The method of claim 1, wherein the therapeutic small protein comprises a growth factor or a proteoglycan.

3. The method of claim 2, wherein the therapeutic small protein comprises insulin-like growth factor 1 (IGF-1) or agrin.

4. The method of claim 1, wherein the counter ion polymer is selected from dextran sulfate (DS), heparin, heparan sulfate, hyaluronic acid, and combinations thereof.

5. (canceled)

6. The method of claim 1, wherein the amphiphilic block copolymer is selected from poly(ethylene glycol)-block-poly(D,L-lactic acid) (PEG-b-PDLLA), poly(ethylene glycol)-block-poly(ε-caprolactone) (PEG-b-PCL), poly(ethylene glycol)-block-poly(lactic-co-glycolic acid) (PEG-b-PLGA) and combinations thereof.

7. (canceled)

8. The method of claim 6, wherein the amphiphilic block copolymer comprises PEG10k-b-PCL40k.

9. The method of claim 1, wherein the therapeutic small protein comprises IGF-1, the counter ion polymer comprises dextran sulfate, and the amphiphilic block polymer comprises poly(ethylene glycol)-block-poly(ε-caprolactone) (PEG-b-PCL).

10. The method of claim 1, wherein the flash nanoprecipitation (FNP) is achieved through a multi-inlet vortex mixer.

11. The method of claim 1, wherein:
(a) the assembling of the polyelectrolyte complex is conducted at a pH from about 1 to about 5;
(b) each jet has a flow rate ranging from about 1 mL/min to about 60 mL/min; and/or
(c) the polyelectrolyte complex has a mass ratio of counter ion polymer to therapeutic small protein of between about 0.1 to about 10.

12-15. (canceled)

16. The method of claim 1, wherein the polyelectrolyte complex is suspended in dimethyl sulfoxide (DMSO).

17. The method of claim 1, wherein the amphiphilic block copolymer is in a water-miscible solvent.

18. The method of claim 17, wherein the water-miscible solvent is selected from acetonitrile (ACN), dimethylformamide (DMF), dimethyl sulfoxide (DMSO), and tetrahydrofuran (THF).

19. (canceled)

20. The method of claim 1, wherein:
(a) the one or more nanoparticles have a ratio of the amphiphilic block copolymer to the small therapeutic protein of about 0.1 to 10;
(b) a concentration of the polyelectrolyte complex is about 2 mg/ml;
(c) a concentration of the amphiphilic block copolymer is about 10 mg/ml;
(d) the one or more nanoparticles have an encapsulation efficiency of therapeutic small protein in the amphiphilic block copolymer is between about 60% to about 99%; and/or
(e) the FNP process has an organic solvent to water ratio of about 0.1 to about 2.

21-24. (canceled)

25. A nanoparticle prepared by the method of claim 1.

26-33. (canceled)

34. The nanoparticle of claim 25, wherein the one or more nanoparticles have:
(a) an average hydrodynamic size of between about 20 nm and about 200 nm;
(b) a polydispersity index of between about 0.05 to about 0.5;
(c) an average zeta potential of between about −5 mV to about −40 mV; and/or
(d) an average loading level of the therapeutic small protein in the nanoparticle is about 0.1 to 50% by weight.

35-38. (canceled)

39. The nanoparticle of claim 25, wherein the nanoparticle is biodegradable.

40. A composition comprising a nanoparticle of claim 25 and a hydrogel, wherein the nanoparticle is distributed throughout the hydrogel.

41. The composition of claim 40, wherein the hydrogel comprises a fibrin gel or a nanofiber-hyaluronic acid hydrogel composite (NHC).

42. A method for treating peripheral nerve injury, the method comprising administering a nanoparticle of claim 25 or a hydrogel composition thereof to a subject in need of treatment thereof.

43. The method of claim 42, wherein the method comprises administering the nanoparticle or composition to one or more of a denervated muscle, an injured nerve tissue, near an injured nerve tissue of the subject.

44. The method of claim 43, wherein the nanoparticle or composition is administered to at least one of the denervated muscle, the injured nerve tissue, near the injured nerve during or after surgical repair of the denervated muscle or injured nerve.

45. The method of claim 42, wherein the administering comprises a controlled release of the small therapeutic protein from the nanoparticle or composition.

46. The method of claim 42, further comprising interval re-dosing the subject with the nanoparticle or composition.

47. The method of claim 46, wherein the interval re-dosing is conducted under ultrasound guidance.

48. The method of claim 42, wherein the subject after being administered the nanoparticle or composition exhibits an improved motor recovery.

49. The method of claim 48, wherein the motor recovery is evidenced through neuromuscular reinnervation, nerve regeneration, a decrease in Schwann cell (SC) senescence, axonal growth, an amelioration of denervation-induced muscle atrophy, an increase in Schwann cell proliferation, an increase in grip strength, and combinations thereof.

50. The method of claim 49, wherein the amelioration of denervation-induced muscle atrophy is evidenced by an increase in mean myofiber cross-sectional area.

51-52. (canceled)
